SEQUENCE LISTING (25 sequences)

SEQ ID NO: 1 - DNA, 924 bp, Homo sapiens
atggggggcctgacagcctcggacgtacacccgaccctgggggtccagctcttctcagctggaatagcggcgtgcttggcggacgtgatcaccttcccgctggacacggccaaagtccggctccaggtccaaggtgaatgcccgacgtccagtgttattaggtataaaggtgtcctgggaacaatcaccgctgtggtaaaaacagaagggcggatgaaactctacagcgggctgcctgcggggcttcagcggcaaatcagctccgcctctctcaggatcggcctctacgacacggtccaggagttcctcaccgcagggaaagaaacagcacctagtttaggaagcaagattttagctggtctaacgactggaggagtggcagtattcattgggcaacccacagaggtcgtgaaagtcagacttcaagcacagagccatctccacggaatcaaacctcgctacacggggacttataatgcgtacagaataatagcaacaaccgaaggcttgacgggtctttggaaagggactactcccaatctgatgagaagtgtcatcatcaattgtacagagctagtaacatatgatctaatgaaggaggcctttgtgaaaaacaacatattagcagatgacgtcccctgccacttggtgtcggctcttatcgctggattttgcgcaacagctatgtcctccccggtggatgtagtaaaaaccagatttattaattctccaccaggacagtacaaaagtgtgcccaactgtgcaatgaaagtgttcactaacgaaggaccaacggctttcttcaaggggttggtaccttccttcttgcgacttggatcctggaacgtcattatgtttgtgtgctttgaacaactgaaacgagaactgtcaaagtcaaggcagactatggactgtgccacataa

SEQ ID NO: 2 - protein, 307 aa, Homo sapiens
MGGLTASDVHPTLGVQLFSAGIAACLADVITFPLDTAKVRLQVQGECPTSSVIRYKGVLGTITAVVKTEGRMKLYSGLPAGLQRQISSASLRIGLYDTVQEFLTAGKETAPSLGSKILAGLTTGGVAVFIGQPTEVVKVRLQAQSHLHGIKPRYTGTYNAYRIIATTEGLTGLWKGTTPNLMRSVIINCTELVTYDLMKEAFVKNNILADDVPCHLVSALIAGFCATAMSSPVDVVKTRFINSPPGQYKSVPNCAMKVFTNEGPTAFFKGLVPSFLRLGSWNVIMFVCFEQLKRELSKSRQTMDCAT

SEQ ID NO: 3 - DNA, 1733 bp, Artificial Sequence
  CAG Promoter
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 4 - DNA, 798 bp, Artificial Sequence
  CBh Promoter
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 5 - DNA, 230 bp, Artificial Sequence
  TTR Promoter
gtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattctt

SEQ ID NO: 6 - DNA, 255 bp, Artificial Sequence
  hAAT Promoter
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 7 - DNA, 72 bp, Artificial Sequence
  CRM8 enhancer
ggggaggctgctggtgaatattaaccaaggtcacccccagttatcggaggagcaaacagggactaagtccac

SEQ ID NO: 8 - DNA, 192 bp, Artificial Sequence
  HCR enhancer
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 9 - DNA, 92 bp, Artificial Sequence
  MVM intron
aagaggtaagggtttaagggatggttggttggtggggtattaatgtttaattacctggagcacctgcctgaaatcactttttttcaggttgg

SEQ ID NO: 10 - DNA, 626 bp, Homo sapiens
gtagctaggcagaggggtaagacaaggggtctcaggacagaggggacgctgttgcgtgcattccatttattctctgctttggtgtaaccactgtttctaggtagggtaggtgaccttccaaagcagtctggccttgtcccagggctggtgctttaggatgggaaactggaactttttctgggattagctgaagaaccaccagggccacagagaatgggttgaccatgactactaccaaattctcccaaaatttagggtgcacttagtattttaagagctgagaatattggcctctcctgagtttactagtcaggtgctttttcctttctttgattcttcgggggttctgtcctatcctactgccctaggggttctggagagttcctggggagggggatattcaaaatgtgcattgtagccagcctccctccatctgcgcgtgagcgaacacacacacacacacacacacacacacacacacacacacacacacacggtagagggaggtggatggaagaggaatgttgctgagaaaagaaacggaaaataggaacacagggggaaatcttggcttaagagtgaactcaatttcgctcccttctgttctgcacctttcttatttccag

SEQ ID NO: 11 - DNA, 593 bp, Artificial Sequence
  Woodchuck Hepatitis Virus posttranscriptional regulatory element
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 12 - DNA, 924 bp, Artificial Sequence
  Codon optimised UCP1 gene
atgggtggattgacagcttcagacgtacatcctacactcggtgtgcagttgtttagcgccggcattgcagcttgtctcgccgatgtcatcacatttcctcttgatacagctaaggtgagacttcaagtgcagggagaatgccccacatcaagtgtgataaggtacaaaggcgtactgggaacgatcaccgcggtagtgaagaccgagggcaggatgaagctgtacagtggactcccagccggactccaacggcagatatccagtgcctccttgaggattggcctttatgacaccgtgcaggaatttctgacagctggcaaggaaaccgccccctcattgggtagcaagatcttggcaggactgactaccggtggtgtggcagtctttatcggacaacctactgaagtcgtgaaggtacgccttcaagcgcaatctcacctgcacggtataaaacccagatacaccggtacttataacgcctaccggattatcgctacaacggaaggactgaccggtctgtggaaagggaccacacccaatcttatgaggtctgttattattaactgcaccgaactcgtaacgtacgatctgatgaaggaagcctttgtaaaaaacaacatcctggctgacgatgtgccatgtcatctggtgagtgcgctcatagccggattctgcgcgacagccatgtcaagccctgtagacgtggtcaagacgaggttcatcaactccccccccggacagtacaaaagcgtgcctaattgcgctatgaaagtcttcactaacgaaggcccgaccgcctttttcaagggccttgtgccaagctttctccgcctcgggagttggaacgtgattatgtttgtgtgctttgaacagctgaagagggagctgtccaaatcccggcagacaatggattgtgctacgtaa

SEQ ID NO: 13 - DNA, 5122 bp, Artificial Sequence
  Vector 1A
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatggggggcctgacagcctcggacgtacacccgaccctgggggtccagctcttctcagctggaatagcggcgtgcttggcggacgtgatcaccttcccgctggacacggccaaagtccggctccaggtccaaggtgaatgcccgacgtccagtgttattaggtataaaggtgtcctgggaacaatcaccgctgtggtaaaaacagaagggcggatgaaactctacagcgggctgcctgcggggcttcagcggcaaatcagctccgcctctctcaggatcggcctctacgacacggtccaggagttcctcaccgcagggaaagaaacagcacctagtttaggaagcaagattttagctggtctaacgactggaggagtggcagtattcattgggcaacccacagaggtcgtgaaagtcagacttcaagcacagagccatctccacggaatcaaacctcgctacacggggacttataatgcgtacagaataatagcaacaaccgaaggcttgacgggtctttggaaagggactactcccaatctgatgagaagtgtcatcatcaattgtacagagctagtaacatatgatctaatgaaggaggcctttgtgaaaaacaacatattagcagatgacgtcccctgccacttggtgtcggctcttatcgctggattttgcgcaacagctatgtcctccccggtggatgtagtaaaaaccagatttattaattctccaccaggacagtacaaaagtgtgcccaactgtgcaatgaaagtgttcactaacgaaggaccaacggctttcttcaaggggttggtaccttccttcttgcgacttggatcctggaacgtcattatgtttgtgtgctttgaacaactgaaacgagaactgtcaaagtcaaggcagactatggactgtgccacataatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 5122 bp, Artificial Sequence
  Vector 1B
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 5748 bp, Artificial Sequence
  Vector 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtagctaggcagaggggtaagacaaggggtctcaggacagaggggacgctgttgcgtgcattccatttattctctgctttggtgtaaccactgtttctaggtagggtaggtgaccttccaaagcagtctggccttgtcccagggctggtgctttaggatgggaaactggaactttttctgggattagctgaagaaccaccagggccacagagaatgggttgaccatgactactaccaaattctcccaaaatttagggtgcacttagtattttaagagctgagaatattggcctctcctgagtttactagtcaggtgctttttcctttctttgattcttcgggggttctgtcctatcctactgccctaggggttctggagagttcctggggagggggatattcaaaatgtgcattgtagccagcctccctccatctgcgcgtgagcgaacacacacacacacacacacacacacacacacacacacacacacacacggtagagggaggtggatggaagaggaatgttgctgagaaaagaaacggaaaataggaacacagggggaaatcttggcttaagagtgaactcaatttcgctcccttctgttctgcacctttcttatttccaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 5214 bp, Artificial Sequence
  Vector 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctaagaggtaagggtttaagggatggttggttggtggggtattaatgtttaattacctggagcacctgcctgaaatcactttttttcaggttggagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 5807 bp, Artificial Sequence
  Vector 6
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctaagaggtaagggtttaagggatggttggttggtggggtattaatgtttaattacctggagcacctgcctgaaatcactttttttcaggttggagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 5145 bp, Artificial Sequence
  Vector 7
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggggaggctgctggtgaatattaaccaaggtcacccccagttatcggaggagcaaacagggactaagtccacgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctaagtgccgggcaatctgggcttaacgggtcctccctgcccgagcaagaggaagggacgctcacctttgagctgctccacagcgccgcctctgcactggcactacctagcccaggtggctctgcaggagtccgaagtcgcgggtttcgtgcccgcatcaggcaacagtgccactgttgtcttcagggctgagtccttttgttcttgcactcacgcctctctgccctccaagccaccatggtgaacccgactacatcagaggttcagcctacaatgggggtaaagatcttttccgctggtgtgtccgcatgcctggctgacatcatcaccttccctttggatacagcaaaggttagactccagattcagggagaaggtcaggccagtagtacaatacgatacaaaggggttttggggactattacaacgctggccaaaaccgagggactgcccaagctctactccggactgcctgcagggatacaaaggcagatttcttttgccagcctgaggattggattgtacgacagcgttcaggagtatttctcttctggaagggaaacacccgcaagcctgggtaacaaaatctctgcgggattgatgaccggtggagtggcagtgtttattggtcagccaacggaggtggtcaaagtgcgaatgcaagcgcaatctcacctgcatggtattaagccgagatatacaggaacatacaacgcatatagagtaattgctaccacagaaagtctcagtacactgtggaagggaacgacacccaacctgatgagaaacgttatcatcaattgcactgagctggtaacttacgatcttatgaagggggcactggtgaacaacaaaattctcgccgacgacgtgccttgtcacctcctcagcgcccttgtggctgggttttgcacaaccctccttgcatcaccagtagatgtcgtcaagacccgatttatcaactccctgccgggacaatatcctagtgtgccgagttgcgcaatgtctatgtatactaaagaaggacccactgccttcttcaaagggtttgttgcaagcttcttgcggctgggctcttggaatgttataatgttcgtgtgtttcgagcaactgaagaaagagctcatgaaatccaggcagacagtcgactgtacgacctaagcaacttggaggaagagatactgaacatcattgggcttctatgctgggagaccacgaataaaaccaaccaaagaaatcaaatgaacagctccgttgactttatttacattacaagatcatttccagtagagagttttgaaacctcttttaattttttttaaagggaaaactaacacatacacatagtttttattcttactgtcttaaagacagaagagcatagcattcactaatattttgagaaaataatacctatataaagtcctgtatttaactggtctttggggagaggtgggagtgtatgactgggtataaagaattctgattacagctcaaactagtgggaaggaaaaattagtccaaaaccctttacatcgataaacactttaaaaaagaaagctatcaaaaaaatattgccatttcatcttatttattgaccacagttcacagctaatatactcaataaagtattgctaattccatctacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 5150 bp, Artificial Sequence
  Vector 8
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgagccaatgaaatacaaagatgagtctagttaataatctacaattattggttaaagaagtatattagtgctaatttccctccgtttgtcctagcttttctcgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 5776 bp, Artificial Sequence
  Vector 9
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgagccaatgaaatacaaagatgagtctagttaataatctacaattattggttaaagaagtatattagtgctaatttccctccgtttgtcctagcttttctcgtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtagctaggcagaggggtaagacaaggggtctcaggacagaggggacgctgttgcgtgcattccatttattctctgctttggtgtaaccactgtttctaggtagggtaggtgaccttccaaagcagtctggccttgtcccagggctggtgctttaggatgggaaactggaactttttctgggattagctgaagaaccaccagggccacagagaatgggttgaccatgactactaccaaattctcccaaaatttagggtgcacttagtattttaagagctgagaatattggcctctcctgagtttactagtcaggtgctttttcctttctttgattcttcgggggttctgtcctatcctactgccctaggggttctggagagttcctggggagggggatattcaaaatgtgcattgtagccagcctccctccatctgcgcgtgagcgaacacacacacacacacacacacacacacacacacacacacacacacacggtagagggaggtggatggaagaggaatgttgctgagaaaagaaacggaaaataggaacacagggggaaatcttggcttaagagtgaactcaatttcgctcccttctgttctgcacctttcttatttccaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 5267 bp, Artificial Sequence
  Vector 10
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 5893 bp, Artificial Sequence
  Vector 11
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtagctaggcagaggggtaagacaaggggtctcaggacagaggggacgctgttgcgtgcattccatttattctctgctttggtgtaaccactgtttctaggtagggtaggtgaccttccaaagcagtctggccttgtcccagggctggtgctttaggatgggaaactggaactttttctgggattagctgaagaaccaccagggccacagagaatgggttgaccatgactactaccaaattctcccaaaatttagggtgcacttagtattttaagagctgagaatattggcctctcctgagtttactagtcaggtgctttttcctttctttgattcttcgggggttctgtcctatcctactgccctaggggttctggagagttcctggggagggggatattcaaaatgtgcattgtagccagcctccctccatctgcgcgtgagcgaacacacacacacacacacacacacacacacacacacacacacacacacggtagagggaggtggatggaagaggaatgttgctgagaaaagaaacggaaaataggaacacagggggaaatcttggcttaagagtgaactcaatttcgctcccttctgttctgcacctttcttatttccaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 6553 bp, Artificial Sequence
  Vector 12
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagccaccatgggcggtcttacagcttcagacgttcatccaacactgggcgttcagctcttctctgccggtatcgccgcctgtttggccgacgtaattactttccccttggacactgctaaagtcagactgcaggtccagggagaatgccctacaagcagcgtgattcggtacaagggcgtgcttgggacaattacagccgtggtgaaaactgaggggcggatgaagctgtactccggactcccggcagggttgcaaaggcagatctctagcgcatccctgcgcatcggcttgtacgataccgtgcaagagttcctcaccgccggcaaagaaaccgccccaagcctgggtagcaagattcttgctggactgacaactggcggtgtggctgtctttattggtcagccgaccgaggtggttaaggtccgcctccaagcacagtcccatctccacggaatcaagcctagatacaccggcacatataacgcttaccggatcattgctaccaccgaggggctcacaggtctgtggaagggaactactcctaacctcatgcgctcagtgatcattaattgcaccgaactcgtcacctatgacctcatgaaagaggccttcgtaaagaacaatatccttgcagacgatgtgccctgccacctcgtaagcgctctgattgcaggtttttgcgccactgctatgtcatccccagtggatgtcgtaaaaacaaggttcatcaacagcccacctggtcagtataagtctgtgccaaactgcgcaatgaaggtcttcactaacgagggcccaaccgcattcttcaaagggttggtgcctagttttttgagactgggcagctggaatgtcatcatgttcgtgtgtttcgaacagctgaagagggagctttctaagagcaggcagacaatggactgtgccacttaatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 5118 bp, Artificial Sequence
  Vector 13
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 775 bp, Homo sapiens
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaag

(19) United States
(12) Patent Application Publication
(10) Pub. No.: US 2023/0330266 A1
(43) Pub. Date: Oct. 19, 2023

(54) GENE THERAPY FOR LIPODYSTROPHY

(71) Applicant: CombiGene AB, Lidingö (SE)

(72) Inventors: Annika Eriksson, Stockholm (SE); Björn Eriksson, Umeå (SE); Stefan Nilsson, Umeå (SE)

(21) Appl. No.: 18/022,473

(22) PCT Filed: Aug. 24, 2021
(86) PCT No.: PCT/GB2021/052197

§ 371 Date: Feb. 21, 2023

(30) Foreign Application Priority Data
Aug. 24, 2020 (GB) 2013194.2

(51) Int. Cl. A61K 48/00; C07K 14/47; C12N 15/86; A61P 3/06

(52) U.S. Cl. CPC A61K 48/0058; C07K 14/47; C12N 15/86; A61P 3/06

(57) ABSTRACT

There is disclosed vectors for treating lipodystrophy and vectors for use in the treatment of lipodystrophy. In particular, the disclosure relates to gene therapy vectors capable of expressing UCP1 for the treatment of lipodystrophy.

### FIELD OF THE INVENTION

The present invention relates to vectors for treating lipodystrophy and vectors for use in the treatment of lipodystrophy. In particular, it relates to gene therapy vectors capable of expressing UCP1.

### BACKGROUND TO THE INVENTION

Lipodystrophy refers to a group of genetic or acquired disorders in which the body is unable to produce and maintain healthy fat tissue [1]. The condition is characterised by abnormal or degenerative conditions of the body's adipose tissue. Lipodystrophy may be either congenital such as congenital generalized lipodystrophy (Beradinelli-Seip syndrome), familial partial lipodystrophy, marfanoid-progeroid-lipodystrophy syndrome or CANDLE syndrome or it may be acquired such as acquired partial lipodystrophy (Barraquer-Simons syndrome), acquired generalized lipodystrophy, centrifugal abdominal lipodystrophy (Lipodystrophia centrifugalis abdominalis infantilis), lipoatrophia annularis (Ferreira-Marques lipoatrophia), localized Lipodystrophy or HIV-associated Lipodystrophy.

Mutations in the genes known as AGPAT2 and BSCL2 account for the majority of cases of congenital generalized lipodystrophy. Mutations in other genes, including CAV1 and PT RF, have also been associated with this form of the disease [2]. Congenital generalized lipodystrophy has an estimated prevalence of only 1 in 10 million people worldwide [2].

Due to an insufficient capacity of subcutaneous adipose tissue to store fat, fat is deposited in non-adipose tissue (lipotoxicity), leading to insulin resistance. Patients display hypertriglyceridemia, severe liver disease and little or no adipose tissue. The average patient lifespan is approximately 30 years, with liver failure being the usual cause of death [3].

There are currently very few treatments available for lipodystrophy. Many patients display low levels of leptin and as such, leptin replacement therapy with human recombinant leptin has been shown to alleviate some of the metabolic complications associated with lipodystrophy [4].

Mitochondrial uncoupling proteins (UCP) are members of the family of mitochondrial anion carrier proteins (MACP). UCPs separate oxidative phosphorylation from ATP synthesis with energy dissipated as heat, also referred to as the mitochondrial proton leak. UCPs facilitate the transfer of anions from the inner to the outer mitochondrial membrane and the return transfer of protons from the outer to the inner mitochondrial membrane. They also reduce the mitochondrial membrane potential in mammalian cells. UCP1, the first UCP to be discovered is expressed predominately in brown adipose tissue (BAT). UCP1 mediated-BAT thermogenesis enhances the oxidation of metabolic substrates necessary for sustaining enhanced thermogenesis. BAT therefore use stored lipids, as well as glucose and triglycerides from the circulation as substrates [5]. UCP1 has been linked to glucose homeostasis [8], metabolic disorders (e.g. WO 2012/092049) and metabolic syndrome [6], but has not been used previously in the treatment of lipodystrophy. Metabolic syndrome is a cluster of conditions that occur together, increasing the risk of heart disease, stroke and type 2 diabetes. These conditions include increased blood pressure, high blood sugar, excess body fat around the waist, and abnormal cholesterol or triglyceride levels. In contrast, lipodystrophy is characterised by a lack of adipose tissue.

Lipodystrophy patients have an unmet need as there are such limited treatment options available. Furthermore, the available options only target the symptoms of the disease.

The present inventors have found that UCP1 could play a role in treating lipodystrophy by normalising liver lipids, restoring hepatic insulin sensitivity and reducing lipoprotein production, and have developed an efficient delivery vector.

### SUMMARY OF THE INVENTION

In a first aspect of the invention, there is provided a vector for treating lipodystrophy, the vector comprising a nucleic acid encoding UCP1, wherein the vector is a lentivirus vector or an AAV vector.

The vector comprises a nucleic acid encoding UCP1. The nucleic acid encodes a functional UCP1 protein. The nucleic acid preferably encodes the human protein, e.g. the wild type human protein.

In particular embodiments, the nucleic acid encoding UCP1 has the nucleotide sequence of SEQ ID NO: 1 or has at least 70% sequence identity thereto, and encodes a functional UCP1 protein. In some embodiments, the nucleic acid encoding UCP1 has the nucleotide sequence of SEQ ID NO: 1 or has at least 72% sequence identity thereto, and encodes a functional UCP1 protein. In a number of embodiments, the nucleic acid encoding UCP1 has the nucleotide sequence of SEQ ID NO: 1 or has at least 74% sequence identity thereto, and encodes a functional UCP1 protein. In other embodiments, the nucleic acid encoding UCP1 has the nucleotide sequence of SEQ ID NO: 1 or has at least 76% sequence identity thereto, and encodes a functional UCP1 protein. In various embodiments, the nucleic acid encoding UCP1 has the nucleotide sequence of SEQ ID NO: 1 or has at least 78% sequence identity thereto, and encodes a functional UCP1 protein. In particular embodiments, the nucleic acid encoding UCP1 has the nucleotide sequence of SEQ ID NO: 1 or has at least 80% sequence identity thereto, and encodes a functional UCP1 protein. In certain embodiments, the nucleic acid encoding UCP1 has the nucleotide sequence of SEQ ID NO: 1 or has at least 82% sequence identity thereto, and encodes a functional UCP1 protein. In various embodiments, the nucleic acid encoding UCP1 has the nucleotide sequence of SEQ ID NO: 1 or has at least 84% sequence identity thereto, and encodes a functional UCP1 protein. In some embodiments, the nucleic acid encoding UCP1 has the nucleotide sequence of SEQ ID NO: 1 or has at least 85% sequence identity thereto, and encodes a functional UCP1 protein. In certain embodiments, the nucleic acid encoding UCP1 has the nucleotide sequence of SEQ ID NO: 1 or has at least 86% sequence identity thereto, and encodes a functional UCP1 protein. In a number of embodiments, the nucleic acid encoding UCP1 has the nucleotide sequence of SEQ ID NO: 1 or has at least 88% sequence identity thereto, and encodes a functional UCP1 protein. In other embodiments, the nucleic acid encoding UCP1 has the nucleotide sequence of SEQ ID NO: 1 or has at least 90% sequence identity thereto, and encodes a functional UCP1 protein. In certain embodiments, the nucleic acid encoding UCP1 has the nucleotide sequence of SEQ ID NO: 1 or has at least 92% sequence identity thereto, and encodes a functional UCP1 protein. In a number of embodiments, the nucleic acid encoding UCP1 has the nucleotide sequence of SEQ ID NO: 1 or has at least 94% sequence identity thereto, and encodes a functional UCP1 protein. In various embodiments, the nucleic acid encoding UCP1 has the nucleotide sequence of SEQ ID NO: 1 or has at least 95% sequence identity thereto, and encodes a functional UCP1 protein. In certain embodiments, the nucleic acid encoding UCP1 has the nucleotide sequence of SEQ ID NO: 1 or has at least 96% sequence identity thereto, and encodes a functional UCP1 protein. In some embodiments, the nucleic acid encoding UCP1 has the nucleotide sequence of SEQ ID NO: 1 or has at least 97% sequence identity thereto, and encodes a functional UCP1 protein. In a number of embodiments, the nucleic acid encoding UCP1 has the nucleotide sequence of SEQ ID NO: 1 or has at least 98% sequence identity thereto, and encodes a functional UCP1 protein. In some embodiments, the nucleic acid encoding UCP1 has the nucleotide sequence of SEQ ID NO: 1 or has at least 99% sequence identity thereto, and encodes a functional UCP1 protein. In particular embodiments, the nucleic acid encoding UCP1 has the nucleotide sequence of SEQ ID NO: 1.

In the embodiments above, the nucleic acid encoding UCP1 may be codon optimised to maximise expression of the protein. In codon optimisation, the amino acid sequence of the encoded protein remains the same so it will still be functional. It is simply the nucleotide sequence that is modified. SEQ ID NO: 12 relates to a codon optimised nucleotide sequence encoding UCP1. This sequence has been found to give a relatively high level of gene and protein expression in human cell-based systems. In some embodiments, the nucleic acid encoding UCP1 has the nucleotide sequence of SEQ ID NO: 12 or has at least 75% sequence identity thereto, and encodes a functional UCP1 protein. In various embodiments, the nucleic acid encoding UCP1 has the nucleotide sequence of SEQ ID NO: 12 or has at least 80% sequence identity thereto, and encodes a functional UCP1 protein. In particular embodiments, the nucleic acid encoding UCP1 has the nucleotide sequence of SEQ ID NO: 12 or has at least 85% sequence identity thereto, and encodes a functional UCP1 protein. In certain embodiments, the nucleic acid encoding UCP1 has the nucleotide sequence of SEQ ID NO: 12 or has at least 90% sequence identity thereto, and encodes a functional UCP1 protein. In various embodiments, the nucleic acid encoding UCP1 has the nucleotide sequence of SEQ ID NO: 12 or has at least 91% sequence identity thereto, and encodes a functional UCP1 protein. In some embodiments, the nucleic acid encoding UCP1 has the nucleotide sequence of SEQ ID NO: 12 or has at least 92% sequence identity thereto, and encodes a functional UCP1 protein. In certain embodiments, the nucleic acid encoding UCP1 has the nucleotide sequence of SEQ ID NO: 12 or has at least 93% sequence identity thereto, and encodes a functional UCP1 protein. In a number of embodiments, the nucleic acid encoding UCP1 has the nucleotide sequence of SEQ ID NO: 12 or has at least 94% sequence identity thereto, and encodes a functional UCP1 protein. In various embodiments, the nucleic acid encoding UCP1 has the nucleotide sequence of SEQ ID NO: 12 or has at least 95% sequence identity thereto, and encodes a functional UCP1 protein. In certain embodiments, the nucleic acid encoding UCP1 has the nucleotide sequence of SEQ ID NO: 12 or has at least 96% sequence identity thereto, and encodes a functional UCP1 protein. In some embodiments, the nucleic acid encoding UCP1 has the nucleotide sequence of SEQ ID NO: 12 or has at least 97% sequence identity thereto, and encodes a functional UCP1 protein. In a number of embodiments, the nucleic acid encoding UCP1 has the nucleotide sequence of SEQ ID NO: 12 or has at least 98% sequence identity thereto, and encodes a functional UCP1 protein. In some embodiments, the nucleic acid encoding UCP1 has the nucleotide sequence of SEQ ID NO: 12 or has at least 99% sequence identity thereto, and encodes a functional UCP1 protein. In particular embodiments, the nucleic acid encoding UCP1 has the nucleotide sequence of SEQ ID NO: 12.

In various embodiments, the UCP1 gene encodes a functional UCP1 protein having the protein sequence of SEQ ID NO: 2 or at least 70% sequence identity thereto. In certain embodiments, the functional UCP1 protein has the protein sequence of SEQ ID NO: 2 or at least 75% sequence identity thereto. In various embodiments, the functional UCP1 protein has the protein sequence of SEQ ID NO: 2 or at least 80% sequence identity thereto. In some embodiments, the functional UCP1 protein has the protein sequence of SEQ ID NO: 2 or at least 85% sequence identity thereto. In other embodiments, the functional UCP1 protein has the protein sequence of SEQ ID NO: 2 or at least 90% sequence identity thereto. In a number of embodiments, the functional UCP1 protein has the protein sequence of SEQ ID NO: 2 or at least 95% sequence identity thereto. In some embodiments, the functional UCP1 protein has the protein sequence of SEQ ID NO: 2 or at least 96% sequence identity thereto. In other embodiments, the functional UCP1 protein has the protein sequence of SEQ ID NO: 2 or at least 97% sequence identity thereto. In a number of embodiments, the functional UCP1 protein has the protein sequence of SEQ ID NO: 2 or at least 98% sequence identity thereto. In certain embodiments, the functional UCP1 protein has the protein sequence of SEQ ID NO: 2 or at least 99% sequence identity thereto. In particular embodiments, the functional UCP1 protein has the protein sequence of SEQ ID NO: 2.

Within this specification, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. In a preferred embodiment, the percent identity between two nucleic acid sequences is determined using the sequence alignment software Clone Manager 9 (Sci-Ed software-www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

The UCP1 gene encodes a ‘functional’ protein. This means that the protein, when expressed, has the same function and activity as the wild type human protein. This could easily be determined by one skilled in the art. The protein encoded by the UCP1 gene may be the wild type human protein. The wild type human sequence of the UCP1 protein is well known to those skilled in the art. For example, it can be found on the publically accessible databases of the National Center for Biotechnology Information. Further, the nucleotide sequences which encode this protein (and which would be contained in the vector) could readily be found or determined by a person skilled in the art, for example, using the genetic code which correlates particular nucleotide codons with particular amino acids.

The vector may be a lentivirus vector or an adeno-associated viral (AAV) vector, although in some embodiments, the vector may be any suitable vector for allowing the expression of the nucleic acid encoding UCP1.

The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively). Details of HIV variants may also be found at http://hiv-web.lanl.aov. Details of EIAV variants may be found through http://www.ncbi.nlm.nih.aov.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes which code for proteins other than gag, pol and env. Examples of additional genes include in HIV, one or more of vif, vpr, vpx, vpu, tat, rev and nef. EIAV has (amongst others) the additional gene S2.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein, Ttm, has been identified that is encoded by the first exon of tat spliced to the env coding sequence at the start of the transmembrane protein.

The lentiviral vector of the present disclosure is a recombinant lentiviral vector. As used herein, the term “recombinant lentiviral vector” (RLV) refers to a vector with sufficient genetic information to allow packaging of an RNA genome, in the presence of packaging components, into a viral particle capable of infecting and transducing a target cell. Infection and transduction of a target cell includes reverse transcription and integration into the target cell genome. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication.

Preferably the recombinant lentiviral vector (RLV) of the present disclosure has a minimal viral genome. As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

A minimal lentiviral genome for use in the present disclosure will therefore comprise (5′) R-U5—one or more first nucleotide sequences—(regulatory element—NOI)n-U3-R (3′).

However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included.

The vector may have at least one of the following: the ATG motifs of the gag packaging signal of the wild type viral vector are ATTG motifs; the distance between the R regions of the viral vector is substantially the same as that in the wild type viral vector; the 3′ U3 region of the viral vector includes sequence from an MLV U3 region; and a nucleotide sequence operably linked to the viral LTR and wherein said nucleotide sequence is upstream of an internal promoter and wherein said nucleotide sequence preferably encodes a polypeptide or fragment thereof.

In a preferred embodiment, the system is based on a so-called “minimal” system in which some or all of the additional genes have be removed.

Preferably the lentiviral vector is a self-inactivating vector. In other words the viral promoter is a self-inactivating LTR.

As known in the art, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes.

In one embodiment, the lentiviral vector is derived from a non-primate lentivirus. The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MW) or an equine infectious anaemia virus (EIAV). Non-primate lentiviral-based vectors do not introduce HIV proteins into individuals.

Preferably the vector is an adeno-associated viral (AAV) vector. The adeno-associated viral vector may be a recombinant adeno-associated viral (rAAV) vector. AAV is a member of the family Parvoviridae which is described in Kenneth I. Berns, “Parvoviridae: The Viruses and Their Replication,” Chapter 69 in Fields Virology (3d Ed. 1996). AAV vectors are also described in “Adeno-Associated Virus Vectors. Design and Delivery”, Editor: Castle, Michael J. (ISBN 978-1-4939-9139-6) and “Adeno-Associated Virus (AAV) Vectors in Gene Therapy”, Editors: Berns, Kenneth I. and Giraud, Catherine (ISBN 978-3-642-80207-2).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid (e.g. a polynucleotide encoding UCP1) to be packaged for delivery to a target cell. According to this particular embodiment, the heterologous nucleotide sequence is located between the viral ITRs at either end of the vector genome. In further preferred embodiments, the parvovirus (e.g. AAV) cap genes and parvovirus (e.g. AAV) rep genes are deleted from the template genome (and thus from the virion DNA produced therefrom). This configuration maximizes the size of the nucleic acid sequence(s) that can be carried by the parvovirus capsid.

According to this particular embodiment, the nucleic acid is located between the viral ITRs at either end of the substrate. It is possible for a parvoviral genome to function with only one ITR. Thus, in a gene therapy vector based on a parvovirus, the vector genome is flanked by at least one ITR, but, more typically, by two AAV ITRs (generally with one either side of the vector genome, i.e. one at the 5′ end and one at the 3′ end). There may be intervening sequences between the nucleic acid in the vector genome and one or more of the ITRs.

Generally, the polynucleotide encoding UCP1 will be incorporated into a parvoviral genome located between two regular ITRs or located on either side of an ITR engineered with two D regions.

AAV sequences that may be used in the present invention for the production of AAV gene therapy vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent albeit with certain differences in tropism, and replicate and assemble by practically identical mechanisms. AAV serotype 1, 2, 3, 4, 5, 6, 7, 8 or 9 may be used in the present invention. However, AAV serotypes 3 or 8 are preferred sources of AAV sequences for use in the context of the present invention. The sequences from the AAV serotypes may be mutated or engineered when being used in the production of gene therapy vectors. In some embodiments, non-human primate AAV serotypes may be used such as those described in WO 03/042397; Gao et al., PNAS, vol. 99, no. 18, pp. 11854-11859 (2002); Castle et al., Methods Mol Biol, 1382: 133-149 (2016); Klein et al. Mol Ther., 16(1): 89-96 (2008); Selot et al., Frontiers in Pharmacology, Volume 8, Article 441 (July 2017), Tanguy et al., Frontiers in Molecular Neuroscience, Volume 8, Article 36 (July 2015), all of which are incorporated herein by reference. In particular, non-human primate AAV serotypes designated as rh serotypes can be used such as AAVrh10 and AAVrh43.

Preferably, the AAV ITR sequences for use in the context of the present invention are derived from AAV1, AAV2, AAV4 and/or AAV6. Likewise, the Rep (Rep78 and Rep52) coding sequences are preferably derived from AAV1, AAV2, AAV4 and/or AAV6. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. Hybrid serotypes can also be used such as those described in Grimm et al., J. Virol. 82, 5887-5911 (2008) and Jang et al. Frontiers in Cellular Neuroscience, Volume 12, Article 157 (June 2018), both of which are incorporated herein by reference.

AAV Rep and ITR sequences are particularly conserved among most serotypes. Moreover, the Rep sequences and ITRs of many AAV serotypes are known to efficiently cross-complement (i.e., functionally substitute) corresponding sequences from other serotypes in production of AAV particles in mammalian cells.

The AAV VP proteins are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, 5 or 8) and the Rep and/or ITR sequences of another AAV serotype (e.g., AAV2). Such pseudotyped rAAV particles are a part of the present invention.

Modified “AAV” sequences also can be used in the context of the present invention, e.g. for the production of AAV gene therapy vectors. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP can be used in place of wild-type AAV ITR, Rep, or VP sequences.

The viral capsid used in the invention may be from any parvovirus, either an autonomous parvovirus or dependovirus, as described above. Preferably, the viral capsid is an AAV capsid (e. g., AAV1, AAV2, AAV3, AAV4, AAV5 or AAV6 capsid). In general, AAV3 capsid or AAV8 capsid are preferred. The choice of parvovirus capsid may be based on a number of considerations as known in the art, e.g., the target cell type, the desired level of expression, the nature of the heterologous nucleotide sequence to be expressed, issues related to viral production, and the like.

Liver-specific expression of a nucleic acid of the invention may advantageously be induced by AAV-mediated transduction of liver cells. Liver is amenable to AAV-mediated transduction, and different serotypes may be used (for example, AAV1, AAV3, AAV5 or AAV8). Hepatocytes are target cells of particular importance in the liver, which makes AAV8 and AAV3 preferred vectors for delivery of the UCP1 polynucleotide.

A parvovirus gene therapy vector prepared according to the invention may be a “hybrid” particle in which the viral ITRs and viral capsid are from different parvoviruses. Preferably, the viral ITRs and capsid are from different serotypes of AAV. Likewise, the parvovirus may have a “chimeric” capsid (e. g., containing sequences from different parvoviruses, preferably different AAV serotypes) or a “targeted” capsid (e. g., a directed tropism).

In the context of the invention “at least one parvoviral ITR nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e., being a recognition site for trans-acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. For safety reasons it may be desirable to construct a parvoviral (AAV) vector that is unable to further propagate after initial introduction into a cell. Such a safety mechanism for limiting undesirable vector propagation in a recipient may be provided by using AAV with a chimeric ITR.

Those skilled in the art will appreciate that the viral Rep protein(s) used for producing an AAV vector of the invention may be selected with consideration for the source of the viral ITRs. For example, the AAV5 ITR typically interacts more efficiently with the AAV5 Rep protein, although it is not necessary that the serotype of ITR and Rep protein(s) are matched.

The ITR(s) used in the invention are typically functional, i.e. they may be fully resolvable and are preferably AAV sequences. Resolvable AAV ITRs according to the present invention need not have a wild-type ITR sequence (e. g., a wild-type sequence may be altered by insertion, deletion, truncation or missense mutations), as long as the ITR mediates the desired functions, e. g., virus packaging, integration, and/or provirus rescue, and the like.

The vector may further comprise a promoter operably linked to the nucleic acid encoding UCP1. The promoter can be any promoter which can drive expression of the UCP1 gene. In some embodiments, the promoter is a liver specific promoter. Liver specific promoters are well known to those skilled in the art, examples of which include the transthyretin (TTR) promoter, alpha-1-antitrypsin promoter (hAAT) and human thyroxine binding globulin (TBG) promoter. In certain embodiments, the promoter is a ubiquitous promoter. Ubiquitous promoters are well known to those skilled in the art, examples of which include CAG, CMV, CBh and SV40.

The promoter may be selected from a TTR, hAAT, TBG, CAG, CMV, CBh or SV40 promoter. In various embodiments, the promoter is selected from a TTR, hAAT, TBG, CAG, CBh or SV40 promoter. In certain embodiments, the promoter is selected from a TTR, hAAT, TBG, CAG or CBh promoter. In some embodiments, the promoter is selected from a TTR, hAAT, TBG or CAG promoter. In further embodiments, the promoter is selected from a TTR, hAAT, CAG or a CBh promoter.

The promoter may be selected from a CAG promoter, a CBh promoter, a transthyretin (TTR) promoter, an alpha-1-antitrypsin promoter (hAAT) or a thyroxine binding globulin (TBG) promoter. It has been found that these promoters provide advantageous results when used to express the UCP1 gene in the liver, particularly when targeting hepatocytes. In some embodiments, the promoter is a CAG promoter. In certain embodiments, the promoter is a CBh promoter. In various embodiments, the promoter is a TTR promoter. In a number of embodiments, the promoter is a hAAT promoter. In several embodiments, the promoter is a TBG promoter.

Examples of the sequences of these promoters are given as SEQ ID NO: 3, SEQ ID NO: 4, SEQ ID NO: 5, SEQ ID NO: 6 and SEQ ID NO: 25. Therefore, in some embodiments, the promoter has a sequence selected from SEQ ID NO: 3, SEQ ID NO: 4, SEQ ID NO: 5, SEQ ID NO: 6 and SEQ ID NO: 25. In particular, the CAG promoter provides a surprisingly high level of expression. In one embodiment, the promoter has the sequence of SEQ ID NO: 3.

The vector may further comprise an enhancer which increases expression of UCP1. The enhancer may be a cis-acting regulatory module (CRM). The CRM is preferably liver specific. Preferably the CRM comprises a conserved transcription factor binding site selected from HNF1, CEBP, LEF-1, FOX, CEBO, IRF, LEF-1/TCF, Ta1β/E47, MyoD and mixtures thereof. The enhancer may be selected from HS-CRM1, HS-CRM2, HS-CRM3, HS-CRM4, HS-CRMS, HS-CRM6, HS-CRM7, HS-CRM8, HS-CRM9, HS-CRM10, HS-CRM11, HS-CRM12, HS-CRM13 and HS-CRM14 [7]. In some embodiments, the enhancer is selected from HS-CRM1 and HS-CRM8. Most preferably the enhancer is from the Serpinal gene. More preferably still, the enhancer is known as HS-CRM8 (hepatocyte specific cis-acting regulatory module 8) [7]. In one embodiment, the enhancer has the sequence of SEQ ID NO: 7.

In an alternative embodiment, the enhancer may be from the human apolipoprotein hepatic control region (HCR). In one embodiment, the enhancer has the sequence of SEQ ID NO: 8. Suitable HCR enhancers are described in Xu et al., J. Cardiovasc. Pharmacol., 65(2):153-9 (2015) which is incorporated herein by reference.

The enhancer is preferably positioned upstream of the promoter. Preferably, the 3′ end of the enhancer is within 100 nucleotides of the 5′ end of the promoter. In some embodiments, the enhancer is adjacent to the promoter, i.e. the 3′ end of the enhancer and the 5′ end of the promoter are adjacent nucleotides.

The vector may further comprise an intron to provide expression benefits. The intron may be an intron from the UCP1 gene, minute virus of mice (MVM), human growth hormone first intron or an intron obtainable from any other suitable gene. In one embodiment, the intron has the sequence of SEQ ID NO: 9. Preferably, the intron is an intron from the UCP1 gene, more preferably intron 1, preferably having the sequence of SEQ ID NO: 10. The intron is preferably located between exons 1 and 2 of the UCP1 gene. When the intron is a minute virus of mice (MVM) intron, it is preferably located before the UCP1 gene, i.e. between the promoter and the UCP1 gene.

The vector may be self-complementary in one embodiment.

The vector may include a posttranscriptional regulatory element. This may be selected from the Woodchuck Hepatitis Virus posttranscriptional regulatory element (WPRE). In one embodiment, the posttranscriptional regulatory element has the sequence of SEQ ID NO: 11.

The vector may further comprise a poly A tail. Preferably, this is positioned downstream of the nucleotide sequence encoding for a functional UCP1 protein. Preferably, the poly A tail is a bovine growth hormone poly A tail. Preferably, this is between 190 and 220 nucleotides in length, more preferably between 200 and 210 nucleotides long.

The vector described above is for treating lipodystrophy. To achieve this, the vector can transduce the key cells of liver, i.e. hepatocytes, but it may also be advantageous to transduce other liver cells, including Kupffer cells.

In one particular embodiment, the vector is an AAV8 vector comprising a nucleic acid encoding UCP1 and a CAG promoter operably linked to the nucleic acid encoding UCP1, the vector further comprising an intron (preferably SEQ ID NO: 10) located between exons 1 and 2 of the UCP1 gene.

In one embodiment, the vector is an AAV8 vector comprising a nucleic acid encoding UCP1 and a CBh promoter operably linked to the nucleic acid encoding UCP1, the vector further comprising an intron (preferably SEQ ID NO: 10) located between exons 1 and 2 of the UCP1 gene. The vector of this embodiment may be self-complementary.

In one embodiment, the vector is an AAV8 vector comprising a nucleic acid encoding UCP1 and a TTR promoter operably linked to the nucleic acid encoding UCP1, the vector further comprising a CRM enhancer and an intron (preferably SEQ ID NO: 10) located between exons 1 and 2 of the UCP1 gene. The vector of this embodiment may be self-complementary.

In one embodiment, the vector is an AAV8 vector comprising a nucleic acid encoding UCP1 and an hAAT promoter operably linked to the nucleic acid encoding UCP1, the vector further comprising an HCR enhancer and an intron (preferably SEQ ID NO: 10) located between exons 1 and 2 of the UCP1 gene.

In some embodiments, there is provided a vector for treating lipodystrophy, the vector comprising a promoter operably linked to a nucleic acid encoding UCP1, wherein the vector is a lentivirus vector or an AAV vector and wherein the promoter is a liver specific promoter.

In other embodiments, there is provided a vector for treating lipodystrophy, the vector comprising a promoter operably linked to a nucleic acid encoding UCP1, wherein the vector is a lentivirus vector or an AAV vector and wherein the promoter is selected from a TTR, hAAT, TBG, CAG, CBh or SV40 promoter.

In certain embodiments, there is provided a vector for treating lipodystrophy, the vector comprising a nucleic acid encoding UCP1, wherein the vector is an AAV8 vector.

In various embodiments, there is provided a vector for treating lipodystrophy, the vector comprising a nucleic acid encoding UCP1, wherein the vector is a lentivirus vector or an AAV vector and wherein the vector further comprises an enhancer.

In a number of embodiments, there is provided a vector for treating lipodystrophy, the vector comprising a nucleic acid encoding UCP1, wherein the vector is a lentivirus vector or an AAV vector and wherein the vector further comprises an intron from the UCP1 gene.

In the embodiments referred to above, the various features of the vector as described earlier in this section are equally applicable to these embodiments and it is intended that they be combined to produce further embodiments.

In another aspect, the invention provides a nucleic acid comprising a promoter operable linked to a polynucleotide sequence encoding UCP1. The description above relating to the vector of the invention is equally applicable to this aspect. For example, the polynucleotide sequence encoding UCP1 may have the nucleotide sequence of SEQ ID NO: 1 or SEQ ID NO: 12, or a sequence having identity to these sequences, as defined above. The polynucleotide sequence encoding UCP1 encodes a functional UCP1 protein and may have the protein sequence of SEQ ID NO: 2 or sequence identity thereto, as defined above. The promoter is as defined above. For example, it may be a liver specific promoter or a ubiquitous promoter; or it may be selected from a CAG promoter, a CBh promoter, a transthyretin (TTR) promoter, an alpha-1-antitrypsin promoter (hAAT) or a human thyroxine binding globulin (TBG) promoter, e.g. having a sequence selected from SEQ ID NO: 3, SEQ ID NO: 4, SEQ ID NO: 5, SEQ ID NO: 6 or SEQ ID NO: 25. The nucleic acid may further comprise an enhancer, as defined above. For example, the enhancer may be a cis-acting regulatory module (CRM) or may be from the human apolipoprotein hepatic control region (HCR). The nucleic acid may further comprise an intron, as defined above. For example, the intron may be an intron from the UCP1 gene or minute virus of mice (MVM). In addition, the nucleic acid may include a posttranscriptional regulatory element and/or a poly A tail, both as defined above.

The nucleic acid can also comprise the specific combination of features described above with reference to the vector. For example, the nucleic acid may comprise:
1) a CAG promoter operably linked to the polynucleotide sequence encoding UCP1, wherein an intron is optionally located between exons 1 and 2 of the polynucleotide sequence encoding UCP1;
2) a TTR promoter operably linked to the polynucleotide sequence encoding UCP1, wherein the nucleic acid further comprises a CRM enhancer and an intron is optionally located between exons 1 and 2 of the polynucleotide sequence encoding UCP1; or
3) a hAAT promoter operably linked to the polynucleotide sequence encoding UCP1, wherein the nucleic acid further comprises an HCR enhancer and an intron is optionally located between exons 1 and 2 of the polynucleotide sequence encoding UCP1.

In further aspects of the invention, there is provided the vector for use in therapy, the vector for use in the treatment of lipodystrophy and the use of the vector in the manufacture of a medicament for treating lipodystrophy.

In another aspect of the invention, there is provided a vector for use in the treatment of lipodystrophy, the vector comprising a nucleic acid encoding UCP1.

The vector is for use in the treatment of lipodystrophy. UCP1 is involved in the regulation of BAT thermogenesis which utilises stored lipids as well as circulating glucose and triglycerides. It may therefore be used to treat the inappropriate fat storage which is typical of all forms of lipodystrophy. In one embodiment, the lipodystrophy is partial familial lipodystrophy.

The vector for use comprises a nucleic acid encoding UCP1 as disclosed above in relation to the vector.

In one embodiment, the vector for use is a lentivirus vector or an adeno-associated viral (AAV) vector as disclosed above in relation to the vector. Preferably the vector is an AAV vector as disclosed above.

The vector for use may further comprise a promoter operably linked to the nucleic acid encoding UCP1 as described above in relation to the vector.

The vector for use may further comprise an enhancer which increases expression of UCP1 as described above in relation to the vector.

The vector for use may further comprise an intron as described above in relation to the vector.

The vector for use may be self-complementary in one embodiment.

The vector for use may include a posttranscriptional regulatory element as described above in relation to the vector.

The vector for use may further comprise a poly A tail as described above in relation to the vector.

The vector for use is for treating lipodystrophy. To achieve this, the vector can transduce cells of liver, hepatocytes.

In one embodiment, the vector for use is an AAV8 vector comprising a nucleic acid encoding UCP1 and a TTR promoter operably linked to the nucleic acid encoding UCP1, the vector further comprising a CRM enhancer and an intron (preferably SEQ ID NO: 10) located between exons 1 and 2 of the UCP1 gene. The vector of this embodiment may be self-complementary.

In one embodiment, the vector for use is an AAV8 vector comprising a nucleic acid encoding UCP1 and a CAG promoter operably linked to the nucleic acid encoding UCP1, the vector further comprising an intron (preferably SEQ ID NO: 10) located between exons 1 and 2 of the UCP1 gene.

In one embodiment, the vector for use is an AAV8 vector comprising a nucleic acid encoding UCP1 and a CBh promoter operably linked to the nucleic acid encoding UCP1, the vector further comprising an intron (preferably SEQ ID NO: 10) located between exons 1 and 2 of the UCP1 gene. The vector of this embodiment may be self-complementary.

In one embodiment, the vector for use is an AAV8 vector comprising a nucleic acid encoding UCP1 and an hAAT promoter operably linked to the nucleic acid encoding UCP1, the vector further comprising an HCR enhancer and an intron (preferably SEQ ID NO: 10) located between exons 1 and 2 of the UCP1 gene.

In one aspect of the invention, there is provided the use of the vector as described above in the manufacture of a medicament for treating lipodystrophy.

In further aspects of the invention, there is provided a pharmaceutical composition comprising the vector, or vector for use, as described above and one or more pharmaceutically acceptable excipients. The one or more excipients include carriers, diluents and/or other medicinal agents, pharmaceutical agents or adjuvants, etc.

Acceptable excipients for therapeutic use are well known in the pharmaceutical art, and are described, for example, in Remington's Pharmaceutical Sciences, Mack Publishing Co. (A. R. Gennaro edit. 1985). The choice of pharmaceutical excipient can be selected with regard to the intended route of administration and standard pharmaceutical practice. The pharmaceutical compositions may comprise as, or in addition to, the excipient, any suitable binder, lubricant, suspending agent, coating agent or solubilising agent.

Preservatives, stabilizers and dyes may be provided in the pharmaceutical composition. Examples of preservatives include sodium benzoate, sorbic acid and esters of p-hydroxybenzoic acid. Antioxidants and suspending agents may be also used.

The pharmaceutical composition may also comprise tolerance-promoting adjuvants and/or tolerance promoting cells. Tolerance promoting adjuvants include IL-10, recombinant cholera toxin B-subunit (rCTB), ligands for Toll-like receptor 2 and/or for Toll-like receptor 9, as well as biologics and monoclonal antibodies that modulate immune responses, such as anti-CD3 and co-stimulation blockers, which may be co-administered with the peptide. Tolerance promoting cells include immature dendritic cells and dendritic cells treated with vitamin D3, (1alpha,25-dihydroxy vitamin D3) or its analogues.

In additional aspects of the invention, there is provided a method of treating lipodystrophy comprising administering a therapeutically effective amount of the vector, or vector for use, as described above, to a patient suffering from lipodystrophy.

When lipodystrophy is “treated” in the above method, this means that one or more symptoms of lipodystrophy are ameliorated. It does not mean that the symptoms of lipodystrophy are completely remedied so that they are no longer present in the patient, although in some methods, this may be the case. The method of treating results in one or more of the symptoms of lipodystrophy being less severe than before treatment.

A “therapeutically effective amount” refers to an amount effective, at dosages and for periods of time necessary, to achieve the desired therapeutic result, such as raising the level of functional protein in a subject (so as to lead to a level sufficient to ameliorate the pathologies associated with lipodystrophy).

The method of treatment causes an increase in the level of functional protein in the subject. In some embodiments, the vector causes expression of UCP1 in the liver. In healthy subjects, UCP1 is not normally expressed in the liver. Therefore, the vector causes expression of functional protein where it would not normally occur.

The vector may be administered in any suitable way so as to allow expression of the UCP1 gene in the liver cells. In particular embodiments, a single administration of the vector can be used to provide gene expression to ameliorate the pathologies associated with lipodystrophy.

By way of example, the vector may be formulated to be delivered parenterally in which the vector is formulated in an injectable form, for delivery, by, for example, an intravenous, intramuscular, intraportal or subcutaneous route. For parenteral administration, the vector may be best formulated in a sterile aqueous solution which may contain other substances, for example enough salts or monosaccharides to make the solution isotonic with blood, or detergents known in the art which prevent the vector adhering to the administration device, for example, Pluronics.

Typically, a physician will determine the actual dosage which will be most suitable for an individual subject and it will vary with the disease, age, weight and response of the particular patient. The appropriate dosage can be determined by one skilled in the art.

The vector may be administered at a single point in time. For example, a single injection may be given. In some embodiments, one or more further administrations of the vector can be given.

In another aspect of the invention, there is provided a host cell comprising the vector, or vector for use, as described above. The host may be any suitable host.

As used herein, the term “host” refers to organisms and/or cells which harbour a nucleic acid molecule or a vector of the invention, as well as organisms and/or cells that are suitable for use in expressing a recombinant gene or protein. It is not intended that the present invention be limited to any particular type of cell or organism. Indeed, it is contemplated that any suitable organism and/or cell will find use in the present invention as a host. A host cell may be in the form of a single cell, a population of similar or different cells, for example in the form of a culture (such as a liquid culture or a culture on a solid substrate), an organism or part thereof.

A host cell according to the invention may permit the expression of a nucleic acid molecule of the invention. Thus, the host cell may be, for example, a bacterial, a yeast, an insect or a mammalian cell.

In an additional aspect of the invention, there is provided a transgenic animal comprising cells comprising the vector, or vector for use, as described above. Preferably the animal is a non-human mammal, especially a primate. Alternatively, the animal may be a rodent, especially a mouse or hamster; or may be canine, feline, ovine or porcine.

As discussed above, SEQ ID NO: 12 is a codon optimised nucleotide sequence encoding UCP1. This sequence has been found to give a relatively high level of gene and protein expression in human cell-based systems. Therefore, in a further aspect of the invention there is provided a nucleic acid which has the nucleotide sequence of SEQ ID NO: 12 or has at least 75% sequence identity thereto, and encodes a functional UCP1 protein. In various embodiments, the nucleic acid has the nucleotide sequence of SEQ ID NO: 12 or has at least 80% sequence identity thereto, and encodes a functional UCP1 protein. In particular embodiments, the nucleic acid has the nucleotide sequence of SEQ ID NO: 12 or has at least 85% sequence identity thereto, and encodes a functional UCP1 protein. In certain embodiments, the nucleic acid has the nucleotide sequence of SEQ ID NO: 12 or has at least 90% sequence identity thereto, and encodes a functional UCP1 protein. In various embodiments, the nucleic acid has the nucleotide sequence of SEQ ID NO: 12 or has at least 91% sequence identity thereto, and encodes a functional UCP1 protein. In some embodiments, the nucleic acid has the nucleotide sequence of SEQ ID NO: 12 or has at least 92% sequence identity thereto, and encodes a functional UCP1 protein. In certain embodiments, the nucleic acid has the nucleotide sequence of SEQ ID NO: 12 or has at least 93% sequence identity thereto, and encodes a functional UCP1 protein. In a number of embodiments, the nucleic acid has the nucleotide sequence of SEQ ID NO: 12 or has at least 94% sequence identity thereto, and encodes a functional UCP1 protein. In various embodiments, the nucleic acid has the nucleotide sequence of SEQ ID NO: 12 or has at least 95% sequence identity thereto, and encodes a functional UCP1 protein. In certain embodiments, the nucleic acid has the nucleotide sequence of SEQ ID NO: 12 or has at least 96% sequence identity thereto, and encodes a functional UCP1 protein. In some embodiments, the nucleic acid has the nucleotide sequence of SEQ ID NO: 12 or has at least 97% sequence identity thereto, and encodes a functional UCP1 protein. In a number of embodiments, the nucleic acid has the nucleotide sequence of SEQ ID NO: 12 or has at least 98% sequence identity thereto, and encodes a functional UCP1 protein. In some embodiments, the nucleic acid has the nucleotide sequence of SEQ ID NO: 12 or has at least 99% sequence identity thereto, and encodes a functional UCP1 protein. In particular embodiments, the nucleic acid has the nucleotide sequence of SEQ ID NO: 12.

A skilled person will appreciate that all aspects of the invention, whether they relate to, for example, the vector, the vector for use, the use, the method of treatment or the host cell for example, are equally applicable to all other aspects of the invention. In particular, aspects of the vector or vector for use may have been described in greater detail than in some of the other aspects of the invention, for example, relating to method of treatment. However, the skilled person will appreciate where more detailed information has been given for a particular aspect of the invention, this information is generally equally applicable to other aspects of the invention.

### BRIEF DESCRIPTION OF THE DRAWINGS

The invention will now be described in detail by way of example only with reference to the figures which are as follows:

FIG. 1: Mice were fed a high fat diet (Research Diets #D124929) for two months prior to transduction with UCP1 AAV and GFP AAV. a) UCP1 expression in the liver 24 days after transduction with UCP1 AAV or GFP AAV. mRNA expression was analysed by qRT-PCR. 18s was used to normalize expression levels. Results are expressed as mean±Std (n=4). Reactions were performed in triplicate. b) Triglyceride level in the liver of mice 24 days after transduction with UCP1 AAV or GFP AAV. Triglyceride levels in the Liver measured by GPO-PAP reagent. Results are shown as mean±Std (n=4). * P≤0.05 c) Blood Glucose level in mice at baseline and 24 days after transduction with UCP1 AAV or GFP AAV. Blood Glucose level in baseline and final time point. Results are shown as mean±Std (n=4). * P≤0.05.

FIG. 2: Mice were switched from regular chow to being fed a high fat diet (Research Diets #D124929) at the same day as transduction with UCP1 AAV and GFP AAV. a) qRT-PCR comparing the levels of UCP1 expression in the liver 6 weeks after transduction with UCP1 AAV or GFP AAV. mRNA expression was analysed by qRT-PCR. 18s was used to normalize expression levels. Results are expressed as mean±Std (n=8). Reactions were performed in triplicate. b) OGTT in mice 6 weeks after transduction with UCP1 AAV or GFP AAV. OGTT was done one day before end of the experiment. Results are shown as mean±Std (n=5). Unpaired t test is used for statistics.

FIG. 3: Schematic illustrations of vector constructs used for mRNA expression studies. CGT2opt corresponds to codon optimised human UCP1.

FIG. 4: qRT-PCR comparing the levels of codon optimised UCP1 expression in HepG2 cells transfected with a) construct numbers 1B, 2, 3 and 6, b) and c) constructs numbers 8, 9, 10, 11 and 12 and d) construct numbers 1B, 2, 8, 9, 10, and 11. mRNA expression was analysed by qRT-PCR. YWHAZ was used to normalize expression levels. Results are expressed as mean±Std (n=3). Reactions were performed in triplicate.

FIG. 5: qRT-PCR comparing levels of codon optimised UCP1 expression from previous FIG. 4 to illustrate that inclusion of intron 1 of the UCP1 gene between exons 1 and 2 results in increased UCP1 expression in HepG2 cells.

FIG. 6: Schematic illustrations of vector constructs used for in vivo studies. CGT2opt corresponds to codon optimised human UCP1. Vector numbers 14, 16 and 17 are self-complimentary vectors.

FIG. 7: qRT-PCR comparing the expression of the hUCP1 by vectors 16, 2, 17, 12 and 14 in the target organ, liver, and an array of other organs. A) shows the relative UCP1 mRNA levels in liver. B), C), D) and E) show the relative UCP1 mRNA levels in spleen, heart, kidney and inguinal white adipose tissue (iWAT), respectively. N.B. Each histogram describes relative expression level in that particular organ, with the lowest expression set to 1.

FIG. 8: qRT-PCR comparing CT values of different organs for pooled cDNA from 10 mice of each group. A) shows the amplification curves from the primer set for housekeeping gene RPL32 in the group treated with vector 2. B) illustrates the difference in CT values when cDNA from different organs is analyzed using the hUCP1 primer set. C) shows the amplification curves from the primer set for housekeeping gene RPL32 in the group treated with vector 14. D) illustrates the difference in CT values when cDNA from different organs is analyzed using the hUCP1 primer set.

FIG. 9: Consistent qRT-PCR and Western blot showing the expression levels of UCP1 in the target organ for an array of other organs. Pooled samples from 10 mice in each group in the qRT-PCR as well as in the western blot. *=background band of slightly different molecular size.

FIG. 10: Western blot (UCP1 expression) of isolated liver mitochondria isolated from mice treated with CGT2-14 (similar results were observed with CGT2-2).

FIG. 11: Basal oxygen consumption of liver mitochondria oxidized two types of substrates and isolated from mice treated with CGT2-2 (similar results with CGT2-14).

FIG. 12: Fatty acid sensitivity in liver mitochondria isolated from mice treated with CGT2-2 on HFD (similar results with CGT2-14).

FIG. 13: Triglyceride content in liver tissue in mice treated with CGT2-2 on HFD (similar results were observed with CGT2-14).

FIG. 14: Reduction in delta-body weight for the CGT2-14-treated mice on both diets.

FIG. 15: Body fat mass measured with EchoMRI for CGT2-14-treated mice.

FIG. 16: Reduction in inguinal adipose tissue for CGT2-2-treated mice.

FIG. 17: Experimental protocol for experiments involving injection of vector CGT2-14 into LDL receptor knockout mice.

FIG. 18: Reduced liver triglycerides and plasma triglycerides in LDL receptor knockout mice treated with vector CGT2-14.

### DETAILED DESCRIPTION OF THE INVENTION

### Examples 1 & 2

### 1. Materials and Methods for In Vivo UCP1 Expression Studies

### AAVs

The AAVs, of serotype 8, used in this study, AAV-TBG-h-UCP1 (Cat. No: 227119) at a concentration of 4.8×10¹³ GC/ml and AAV-TBG-EGFP (Cat. No: VB1743) at a concentration of 2.7×10¹³ GC/ml, were supplied by Vector Biolabs, USA and the UCP1 gene was inserted into the vectors identified in the afore-mentioned catalogue numbers. The AAVs were kept at −80° C. and diluted in PBS before performing IV injections.

### Animal Handling

C57BL/6 mice were supplied by Charles River Laboratories Inc. (Charles River Laboratories Inc.,'s-Hertogenbosch, Netherlands). All mice were housed in single cages under a 12-h light/12-h dark cycle. Food and water were provided ad libitum and at the start of the study 1, two months prior to transduction, the standard diet was changed to a high fat diet (Rodent diet with 60 kcal % fat, D12492, Research Diet, USA) and this diet was kept throughout the study. Animal experiments were performed according to laws and regulations as established by the European Union (Directive 2010/63/EU) and the Swedish legislative authorities [Swedish Animal Protection Regulation (1988: 539); Animal Protection Act (SFS 1988:534); Swedish Agricultural Agency's regulations (L150)].

For study 1, the mice were divided into 2 groups (number of mice per group=4) and GFP AAV and UCP1 AAV were injected intravenously as single doses at a concentration of 1×10¹² GC at beginning of experiment. Body composition of the mice were monitored by EchoMRI at day 24. Blood samples were collected at end point of the experiment (24 days after injection) through the tail vein for blood glucose analysis. Livers were collected for gene expression analysis and in vitro measurements of triglycerides.

For study 2, the mice were divided into 3 groups. Each of the treatment groups received intravenous injections (IV) of GFP AAV (n=8) or UCP1 AAV (n=8) respectively, as single doses at a concentration of 0.5×10¹³ GC at the beginning of the experiment. Simultaneously mice were switched from regular chow to being fed a high fat diet (Research Diets #D124929). Vehicle control (n=2) received PBS.

Body composition of the mice was monitored by EchoMRI 5 weeks after transduction. The day before termination, an oral glucose tolerance test was performed.

### Gene Expression

Total RNA was prepared from mouse liver using RNeasy Lipid Tissue Mini kit (Cat No 74804, Qiagen). First strand cDNA was synthesized from equal amount of total RNA with random hexamer primer, DNTP mix and RevertAid H Minus Reverse Transcriptase (REF K0231 Thermo Scientific), treated with Ambion RNase inhibitor (Cat no 00021540 Invitrogen). RNA concentration and quality were determined using NanoDrop 2000 Spectrophotometer (Thermo Fisher). RNA was stored at −80° C. until cDNA synthesis.

For each PCR reaction 10 μl Maxima Probe/ROX qPCR Master Mix (2×), (Cat No. K0232, Thermo Scientific), 2 μl TaqMan Gene expression assay and 105 ng of template cDNA were mixed in a total volume of 20 μl. PCR was carried out in 98-well plates using the StepOnePLus™ Real-time PCR System instrument (Applied Biosystems), and analyzed by StepOne™Software v.2.3. (Applied Biosystems). The Comparative MCt method was used to quantify gene expression and expression of 18s was used to normalize expression levels. All reactions were done in triplicate or duplicate.

The TaqMan Gene expression assays used were: hUCP1 (Hs01084772-ml) and murine 18s (Mm03928990-g1).

### Echo MRI

In study 2, body composition of the mice was measured by an EchoMRI™ body composition analyser at the UCCB (Umeå a Centre for Comparative Biology) facility at day 24. In study 3, measurements were taken 5 weeks after AAV IV injections.

### In Vitro Measurement of Triglyceride Levels in the Liver

Triglyceride levels (TG) were determined using the Triglycerides GPO-PAP reagent (Roche Diagnostics GmbH, Mannheim, Germany). Liver tissue was homogenized in 1 ml of PBS, Chloroform:methanol (2:1) was mixed with the homogenate and centrifuged at 3000 rpm, for 10 minutes in 4° C. The lower organic phase was transferred into new glass tubes and left for 48 hrs to evaporate. The dried lipids were dissolved in 500 μl of 1% Triton-X 100 in Chloroform, mixed and dried again for 24 hrs. The dried lipids were dissolved in 100 μl distilled water.

Human plasma was used as a standard and six different concentrations were used to generate the standard curve: undiluted plasma and plasma dilutions at a ratio of 1/2, 1/4, 1/8, 1/16, 1/32 and 1/64.

The extracted lipid from mouse liver tissues were diluted at a 1/70 ratio. Purified water (Milli-Q water purification system, Merck KGaA, Darmstadt, Germany) was used for dilution of standards and samples.

15 μL of each sample was added in duplicates in a flat-bottom 96-well plate. 50 μL of the GPO-PAP reagent was added to each well and the plate was incubated on a shaker at room temperature for 20 min. Absorbance was measured at 500 nm and 570 nm using a SpectraMax 340PC spectrophotometer (Molecular Devices, LLC. San Jose, CA, USA) and TG values calculated according to the instructions from the kit manufacturer.

### Oral Glucose Tolerance Test

Mice were fasted for 4 hrs before Oral Glucose tolerance test. Mice received 2 g/kg body weight Glucose (Cat nr G-8644, Sigma, USA) through oral gavage. Blood glucose were measured at −5.5, 10, 30, 60 and 120 minutes after glucose gavage through tail vein by glucometer and glucose strips. (Accu-Chek, Roche, Switzerland).

2. Materials and Methods for mRNA Expression Studies

### Constructs

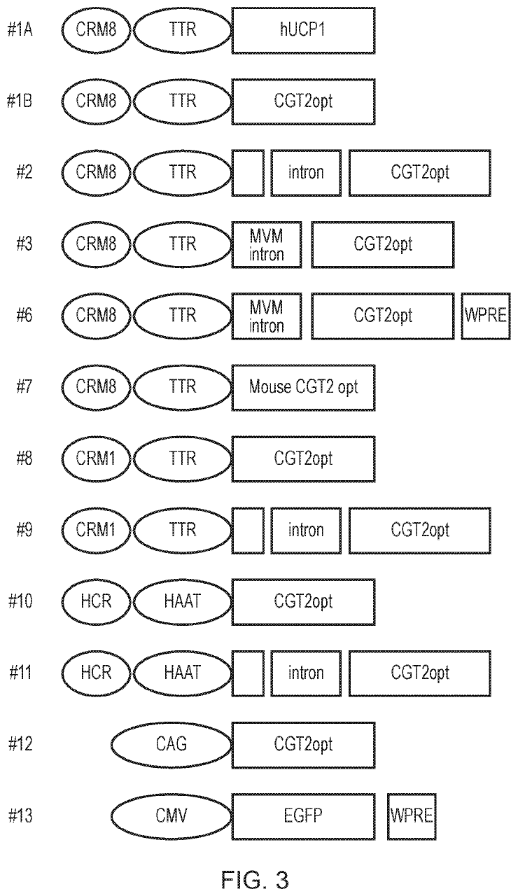

Constructs were custom produced at VectorBuilder. The vectors are provided in Table 1 below and FIG. 3.

TABLE 1
Vectors used for mRNA expression studies

| | SEQ | |
|---|---|---|
| Vector | ID | |
| No. | No. | |
| 1A | 13 | pAAV[Exp]- |
| | | CRM8/TTRminhuman>5′UTR:hUCP1[NM_021833.4]:3′UTR |
| 1B | 14 | pAAV[Exp]-CRM8/TTRminhuman>5′UTR:hCGT2(opt):3′UTR |
| 2 | 15 | pAAV[Exp]-CRM8/TTRminhuman>5′UTR:InhCGT2(opt):3′UTR |
| 3 | 16 | pAAV[Exp]-CRM8/TTRminhuman>MVM |
| | | Intron:5′UTR:hCGT2(opt):3′UTR |
| 6 | 17 | pAAV[Exp]-CRM8/TTRminhuman>MVM |
| | | Intron:5′UTR:hCGT2(opt):3′UTR:WPRE |
| 7 | 18 | pAAV[Exp]-CRM8/TTRminhuman>5′UTR:mCGT2(opt):3′UTR |
| 8 | 19 | pAAV[Exp]-CRM1/TTRminhuman>5′UTR:hCGT2(opt):3′UTR |
| 9 | 20 | pAAV[Exp]-CRM1/TTRminhuman>5′UTR:InhCGT2(opt):3′UTR |
| 10 | 21 | pAAV[Exp]-HCR/hAAT>5′UTR:hCGT2(opt):3′UTR |
| 11 | 22 | pAAV[Exp]-HCR/hAAT>5′UTR:InhCGT2(opt):3′UTR |
| 12 | 23 | pAAV[Exp]-CAG>5′UTR:hCGT2(opt):3′UTR |
| 13 | 24 | pAAV[Exp]-CMV>EGFP:WPRE |

### Plasmid Purification

Qiagen plasmid maxi kit (#12163, Qiagen) 1×TE (10 mM Tris-HCl pH 8.0, 1 mM EDTA), LB medium, Ampicillin 50 mg/ml (TBK, County hospital of Vasterbotten)

### Cell Line and Growth Medium

Human liver hepatocellular carcinoma cell line HepG2 (Cat No. HB-8065) was obtained from LGC Promochem-ATCC. Dulbecco's Modified Eagle Medium (DMEM; Cat No. 21885025), Fetal Bovine Serum (FBS; Cat No. 10500064), Gentamicin (Cat No. 15750060), MEM Non Essential Amino Acids (MEM NEAA; Cat No. 11140035), PBS pH 7.4 (10010023) and Trypsin (Cat No. 25200056) were purchased from Gibco.

### Transfection

Opti-MEM™ I Reduced Serum Medium (Cat No. 31985062, Gibco), Lipofectamine™ 3000 Transfection Reagent, (Cat No. L3000008, Lot no: 2146266/2189652, Invitrogen™), 6-Well Cell Culture Plate, TC-treated (Cat No. 353046, Falcon), PBS pH 7.4, (1×) (Cat No. 10010023, Gibco), TissueLyser LT (Qiagen).

RNA Preparation and cDNA Synthesis

NucleoSpin RNA kit (#740955.50, Macherey-Nagel), 2-mercaptoethanol (#M3148, Sigma), TissueLyser (Qiagen), Stainless Steel Beads 5 mm (Cat No. 69989, Qiagen) NanoDrop 2000 Spectrophotometer (Thermo Scientific). SuperScript™ IV VILO™ Master Mix (Cat No. 11756050, Invitrogen™) and SuperScript™ IV VILO™ Master Mix with ezDNase™ Enzyme (Cat No. 11766050, Invitrogen™).

### Quantitative RT-PCR

Maxima Probe/ROX qPCR Master Mix (2×), (Cat No. K0232, Thermo Scientific), StepOnePLus™ Real-time PCR System instrument (Applied Biosystems), StepOne™Software v.2.3 (Applied Biosystems).

### Gene Expression Assays

TaqMan™ Gene Expression Assays (FAM) (Cat NO. 4331182) and Custom TaqMan™ Gene Expression Assays, FAM (Cat NO. 4331348) were obtained from Applied Biosystems:

| | |
|---|---|
| YWHAZ | Assay ID: hs01122445_g1 |
| UCP1 (Human UCP1) | Assay ID: Hs01084772_m1 |
| hCGT2opt (Human UCP1 | Assay ID: APKA7YP |
| codon optimised) | |
| MOCGT2OPT (Mouse | Assay ID: APT2DW4 |
| UCP1 codon optimised) | |
| EGFP | Assay ID: Mr04097229_mr |

### Plasmid Purification

Constructs were prepared as glycerol stocks and stored at −80° C. Upper phase of glycerol stock was lightly thawed and approximately 15 μl were aspirated and used to inoculate LB liquid medium supplemented with 100 mg/ml Ampicillin. Bacteria cultures were incubated at 37° C. for 16 hours with vigorous shaking (300 rpm). Plasmid purification was performed according to instructions provided by kit supplier (Qiagen). DNA pellet was resuspended in 150 μl 1×TE and stored at −80° C. DNA concentration and quality were determined using NanoDrop 2000 Spectrophotometer.

### Cell Culture and Transfection

Human liver hepatocellular carcinoma cells were cultured in DMEM containing 1 g/L glucose supplemented with 10% heat inactivated FBS, 1 mM MEM NEAA, and 25 μg/ml gentamicin. The cells were incubated in a humidified atmosphere of 95% air and 5% CO₂ at 37° C. The cells were seeded in 6-well plates (9.6 cm²/well) at a density of 500000 cells/well. The plates were cultured overnight in complete DMEM.

Transfection was performed according to the Transfection protocol provided by Transfection reagent supplier. The amount of Lipofectamine used was 3.75 μl/well and the amount of DNA added to each well was 5 μg. Transfections were done approximately 24 hours after seeding. Three transfection experiments were performed, and three biological replicates of each construct was transfected.

### RNA Preparation

Cells were harvested for RNA extraction 47-48 hours after transfection. RNA was prepared by using NucleoSpin RNA kit. Media was removed and the wells were rinsed two times with PBS (5 and 3 ml), and thereafter cells were lysed directly in 350 μl buffer RA1 supplemented with 3.5 μl β-mercaptoethanol. Cell lysates were transferred into 2 ml epp tubes containing a steel bead and were homogenized using a Tissulyser, 30 hz, 30 seconds. RNA samples were purified according to manufacturer's instructions and eluted in a volume of 30 μl RNase-free H₂O. RNA concentration and quality were determined using NanoDrop 2000 Spectrophotometer. RNA was stored at −80° C. until cDNA synthesis.

cDNA Synthesis

cDNA was synthesized by using SuperScript™ IV VILO™ Master Mix or SuperScript™ IV VILO™ Master Mix with ezDNase™ Enzyme kit. For each reaction, 1500-2000 ng of RNA and were added. cDNA synthesis was performed according to protocol instructions supplied by kit provider. After synthesis cDNA was diluted in water to a final concentration of 10 ng/μl and stored at −20° C.

### Quantitative RT-PCR

For each PCR reaction 10 μl Mastermix (2×), 2 μl TaqMan Gene expression assay and 10 ng of template cDNA were mixed in a total volume of 20 μl.

PCR was performed in a StepOnePLus™ Real-time PCR System instrument and analyzed by StepOne™Software v.2.3. The Comparative ΔΔCₜ method was used to quantify gene expression and expression of tyrosine 3-monooxygenase/tryptophan 5-monooxygenase activation protein, zeta polypeptide (YWHAZ) was used to normalize expression levels.

Three biological replicates were analyzed for each construct. All reactions were performed in triplicates and non-template controls were included in each PCR run.

### Results

1. UCP1 is Capable of being Expressed in the Liver and can Decrease Triglyceride Levels

In study 1, no effect on body composition or organ weights was shown in mice receiving UCP1 AAV injections compared with control mice injected with GFP AAV as illustrated in Table 2 below.

TABLE 2
Body, liver, kidney, SC fat and spleen weight were
measured at the end point of experiment (d24).
Fat and lean mass % were measured by EchoMRI
at day 24. Results are shown as mean ± Std
(n = 4). Unpaired t test was used for statistics.

| Physiological Parameter | GFP-AAV | UCP1-AAV |
|---|---|---|
| Body weight(g) | 50.05 ± 0.95 | 51.62 ± 1.84 |
| Liver weight(g) | 2.69 ± 0.35 | 3.15 ± 0.54 |
| Kidney weight(g) | 0.17 ± 0.02 | 0.18 ± 0.03 |
| Subcutaneous fat(g) | 1.50 ± 0.43 | 1.46 ± 0.35 |
| Spleen weight (g) | 0.92 ± 0.06 | 0.94 ± 0.05 |
| Glucose (mmol/L) | 10.40 ± 2.25 | 7.92 ± 0.43 |
| fat mass % | 41.06 ± 1.24 | 40.66 ± 1.22 |
| Lean mass % | 56.21 ± 1.14 | 56.68 ± 1.1 |

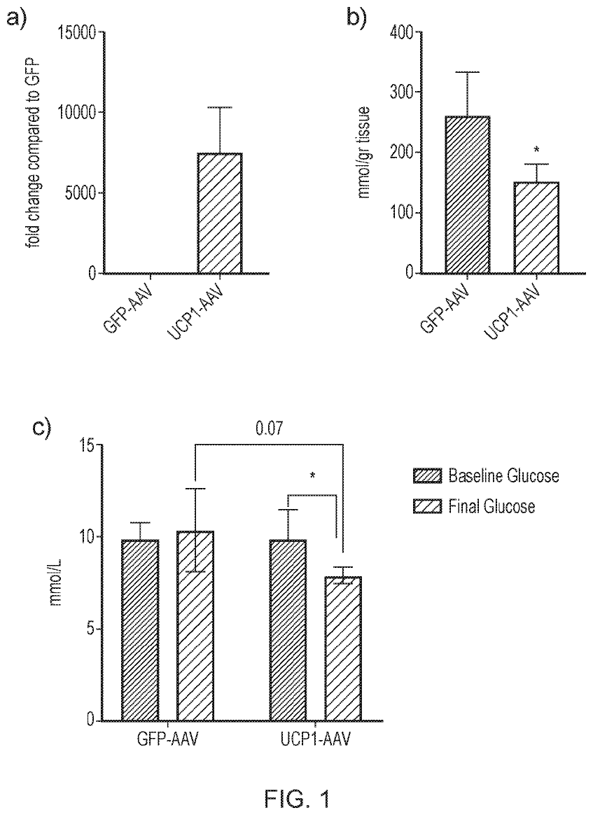

Intravenous injection of UCP1 AAV induced UCP1 gene expression in the liver of wild type mice in study 1. A robust expression of UCP1 was obtained in the liver of mice injected with UCP1 AAV at the end point of experiment (day 24), compared to the GFP control group (FIG. 1a). Expression of UCP1 gene in the liver caused triglyceride levels in the liver to decrease (FIG. 1b) as well as blood glucose levels (FIG. 1c).

In study 2, no effect on body composition or organ weights was shown in mice receiving UCP1 AAV injections compared with control mice injected with GFP AAV as illustrated in Table 3 below.

TABLE 3
Body, liver, SC and PG fat weight were measured at
the end point of experiment. Fat and lean mass % were
measured by EchoMRI one week before the end of
the experiment. Results are shown as mean ± Std
(n = 8 and 2). Unpaired t test was used for statistics.

| Physiological parameter | GFP-AAV | UCP1-AAV | Vehicle |
|---|---|---|---|
| Body weight (g) | 40.21 ± 2.93 | 37.07 ± 4.48 | 41.23 ± 0.09 |
| Liver weight (g) | 1.36 ± 0.14 | 1.35 ± 0.18 | 1.52 ± 0.02 |
| Subcutaneous fat weight (g) | 1.83 ± 0.44 | 1.50 ± 0.54 | 2.40 ± 0.18 |
| Perigonadal fat weight (g) | 2.39 ± 0.52 | 2.00 ± 0.43 | 2.11 ± 0.15 |
| Fat mass final % | 21.51 ± 4.92 | 25.10 ± 4.96 | |
| Lean mass final % | 75.03 ± 6.1 | 70.59 ± 3.8 | |

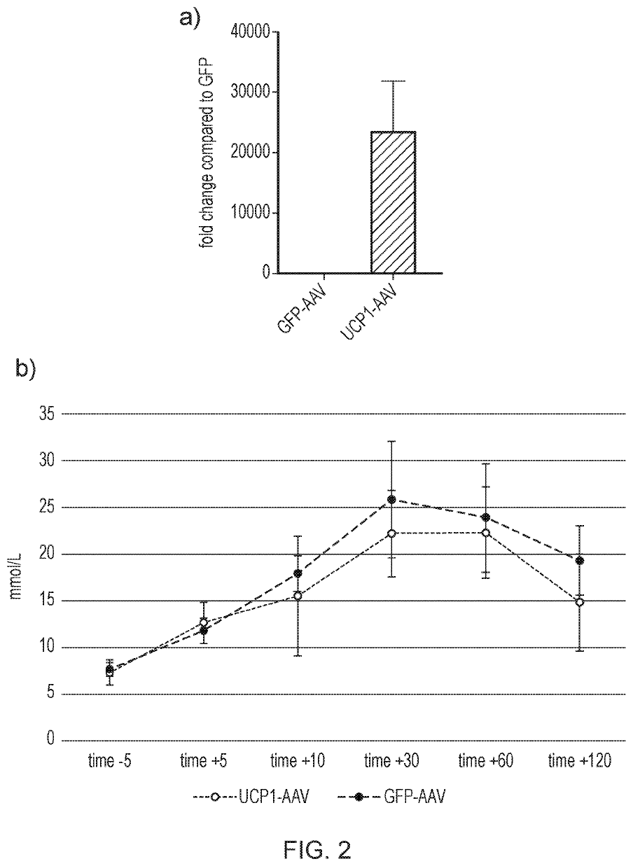

A robust expression of UCP1 was obtained in the liver of mice treated with UCP1 AAV at the end point of experiment (FIG. 2a). The EchoMRI data did not show any differences between groups at the end point of the experiment. Mice receiving UCP1-AAV showed a non-statistically significant improvement of glucose tolerance in OGTT compared to GFP-AAV control group (FIG. 2b). By increasing the dose of AAV, the expression of UCP1 increased respectively. The effect on body weight, body fat composition and glucose tolerance is not significant.

2. mRNA Expression

The expression of the target gene (UCP1) in HepG2 cells transfected with candidate constructs was studied to determine which regulatory elements impacted the expression of UCP1.

12 different constructs were included in the study. Three of those were control vectors: one expressed the EGFP, one codon optimised mouse UCP1 and one the human UCP1 without codon optimisation. The remaining 9 vectors expressed the codon optimised human UCP1, regulated by different promoter/enhancer elements and with or without intron sequences as shown in FIG. 3.

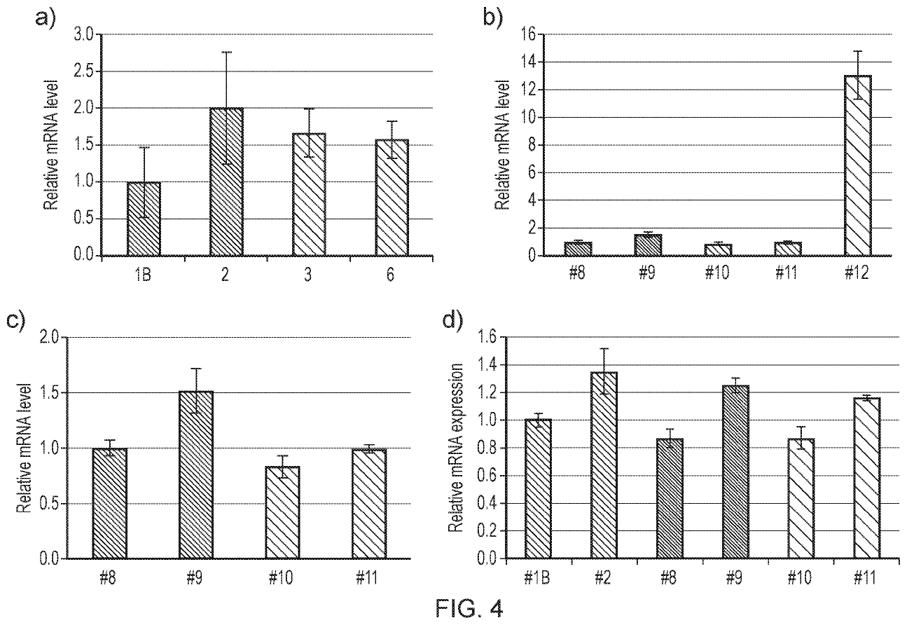

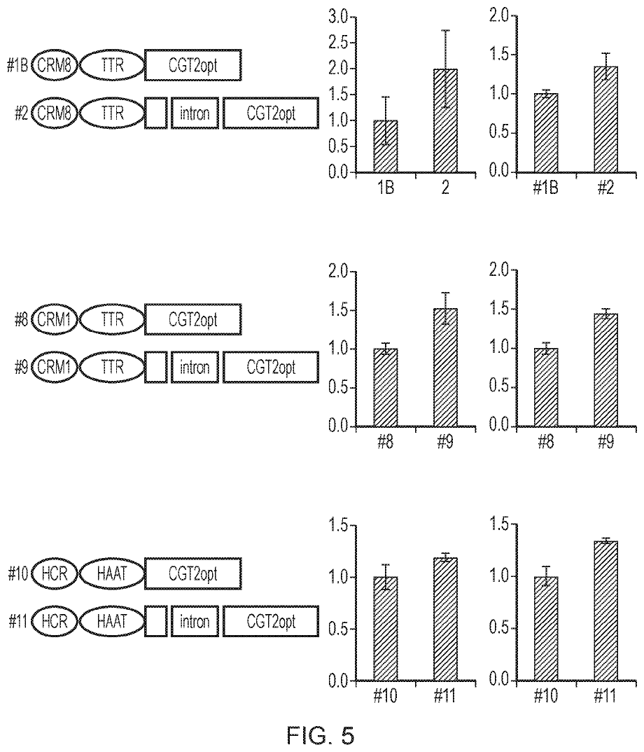

The constructs were transfected into HepG2 cells and cells were harvested 48 hours after transfection. mRNA was prepared and comparative gene expression of target gene (UCP1) was analysed by quantitative RT-PCR as shown in FIG. 4 and Table 4 below.

The comparative mRNA expression analysis in HepG2 cells did detect differences between gene regulatory elements in the different constructs.

A robust expression of UCP1 was obtained from all constructs. The highest level of UCP1 expression was found in cells transfected with the vector in which UCP1 expression was regulated by the CAG promoter.

When comparing the other constructs, a higher UCP1 expression was observed from vectors that had one intron included, either the first endogenous UCP1 intron or MMV intron.

The addition of the WPRE element did not affect UCP1 expression.

Table 4 below shows the CT values from qRT-PCR demonstrating the expression of EGFP, mouse UCP1 optimised and human UCP1 (not codon optimised).

TABLE 4
Results from transfections with construct number
13, 7, 1A are displayed as CT values since different TaqMan ™
Gene Expression Assays were used in the RT-PCR thus inhibiting
direct comparable analysis. The same threshold value was used
for the different assays. Construct number 1B was
included as reference.

| Construct | | CT | |
|---|---|---|---|
| no. | Gene | mean | Std |
| #13 | YWHAZ | 21.65 | 0.52 |
| | EGFP | 12.94 | 0.77 |
| #7 | YWHAZ | 21.34 | 0.11 |
| | Mouse UCP1 optimised | 20.20 | 0.15 |
| #1A | YWHAZ | 21.71 | 0.13 |
| | Human UCP1 | 16.74 | 0.71 |
| #1B | YWHAZ | 21.16 | 0.03 |
| | Human | 18.13 | 0.60 |
| | UCP1 optimised | | |

### In Vivo Experiments

### Example 3

### Expression in Different Tissues

### Materials & Methods

### Animals and Husbandry

Male C57BL/6J Jax® Mice (Stock Number 000664), 8 weeks of age were purchased from Charles River Laboratories (Germany). All animals were housed in the Umeå University animal facility (Umeå Centre for Comparative Biology; UCCB) with a 12:12 h light-dark cycle (lights on at 12.30 p.m.) at a constant temperature of 22° C. The animals were ear marked with a unique identification number, and groups of 5 mice were housed in transparent polycarbonate cages that comply with the requirements of the Code of Practice for the housing and care of animals used in scientific procedures. Wood chips were used as bedding material and environmental enrichment was provided. The animals were allowed ad libitum access to tap water and a standard pelleted diet (CRM(E)Rodent, Special Diets Services, Scanbur BK, Sweden) throughout the accommodation and study period.

Reagents and Material for RNA Preparation and cDNA Analysis

NucleoSpin RNA kit (#740955.50, Macherey-Nagel), RNeasy Fibrous Tissue Mini Kit (Cat. No. 74704, Qiagen), RNeasy Lipid Tissue Mini Kit (Cat. No. 74804, Qiagen), RNase-Free DNase Set (Cat. No. 79254, Qiagen).

2-mercaptoethanol (#M3148, Sigma), NanoDrop 2000 Spectrophotometer (Thermo Scientific). SuperScript™ IV VILO™ Master Mix with ezDNase™ Enzyme (Cat No. 11766050, Invitrogen™).

### Reagents and Material for Quantitative RT-PCR

Maxima Probe/ROX qPCR Master Mix (2×) (Cat No. K0232, Thermo Scientific), StepOnePLus™ Real-time PCR System instrument (Applied Biosystems), StepOne™Software v.2.3 (Applied Biosystems).

Gene Expression Assays for qRT-PCR

TaqMan™ Gene Expression Assays (FAM) (Cat NO. 4331182) and Custom TaqMan™ Gene Expression Assays, FAM (Cat NO. 4331348) were obtained from Applied Biosystems:
RPL32, Assay ID: Mm02528467_g1
hCGT2opt (Human UCP1 codon optimised), Assay ID: APKA7YP

### Reagents and Material for Western Blot Analysis

RIPA lysis and extraction buffer (Cat No. 89900, Thermo Scientific™), Halt protease inhibitor cocktail, EDTA free 100× (Cat No. 87785, Thermo Scientific™), BCA protein assay kit (Cat No. 23225, Pierce), 4-12% Criterion™ XT Bis-Tris Protein Gel (Cat No. 3450124, BioRad), XT MES buffer kit (Cat No. 1610796, BioRad), 2-Mercaptoethanol (Cat. No. M3148 Sigma), Precision Plus Protein™ All Blue Prestained Protein Standards, (Cat No. 1610373, BioRad), Supported Nitrocellulose Membranes (Cat No. 1620070, BioRad), Extra Thick Blot Filter Paper (Cat No. 1703967. BioRad), MeOH (1.06012.2500, Merck), Tris-base (Cat No. T6066, Sigma), Glycine (Cat No. 0167, VWR), NaCl (Cat No. 27810, VWR), Tris pH 7.5 (TBK, County hospital of Vasterbotten), Tween 20 (Cat No. P1379, Sigma), Bovine Serum Albumine (BSA; Cat No. 3912, Sigma), Fat free powdered milk (Semper, Sweden), SuperSignal West Dura Extended Duration Substrate (Cat No. 37071, Thermo Scientific™) Imaging was performed using ChemiDoc imaging system and Image Lab 6.1 (BioRad).

### Antibodies for Western Blot

Anti UCP1 antibody (Cat No. ab10983, Abcam), β-Actin antibody (Cat No. 4967S, Cell Signaling) and Peroxidase AffiniPure Goat Anti-Rabbit IgG (H+L) (Cat No. 111-035-003, Jackson).

### Study Design

A total number of 60 male C57BL/6J mice, 7-8 months old, were assigned into six groups (n=10) based on mean bodyweight/cage and injected intravenously with 1*10¹³ GC/kg of test compounds as single doses at beginning of experiment. Bodyweight and food intake was monitored once a week. After three weeks of treatment mice were euthanized by cervical dislocation and decapitated immediately thereafter, for blood sampling from carotid artery.

Left liver Lobule, Heart, Inguinal white adipose tissue (iWAT), Kidney, Lung, Spleen and Brain were isolated and rapidly frozen in liquid nitrogen and stored at −80° C. until RNA and western lysates were prepared.

### Test Compound Administration

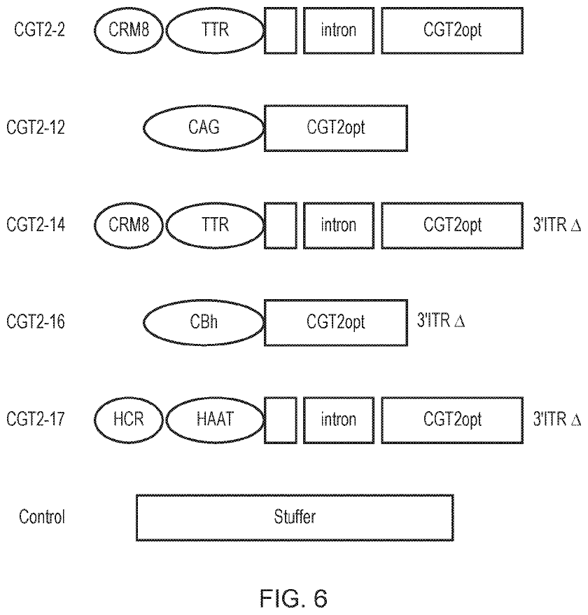

The virus stocks were stored in −80° C., thawed on ice and diluted in cold PBS to a concentration of 5*10¹² GC/ml before injections. Diluted virus stocks were kept on ice until injections. The mice received a dose of 1*10¹³ GC/kg in a volume of 60 μl-100 μl depending on bodyweight. Each vector was injected into 10 mice by i.v. injections into the tail vein. CGT2-2, CGT2-12 and CGT2-14 were injected on one occasion and CGT2-16 and CGT2-17 two weeks later (Vectors shown in FIG. 6). Due to delayed delivery, Control vector was injected 3 months later.

RNA Preparation and cDNA Synthesis

Snap frozen tissues from Left liver Lobule, Heart, Inguinal white adipose tissue (iWAT), Kidney, Lung, Spleen and Brain were crushed in liquid nitrogen to a fine powder using a mortar and a pestle and thereafter homogenized in appropriate buffer.

RNA from left Liver lobule, kidney, Lung and Spleen was prepared using Total RNA Isolation Nucleospin II kit. Tissues were homogenized in 350 μl buffer RA1 supplemented with 3.5 μl β-mercaptoethanol using a tissue homogenizer. RNA from iWAT and Brain was prepared using RNeasy Lipid Tissue Mini Kit. Tissues were homogenized in 1000 μl Qiasol using a tissue homogenizer. RNA from Heart muscle tissues was prepared using RNeasy Fibrous Tissue Mini kit. Tissues were homogenized in 300 μl buffer RLT supplemented with 3.0 μl β-mercaptoethanol using a tissue homogenizer.

Subsequently RNA samples were purified according to manufacturer's instructions and eluted in a volume of 30 μl RNase-free H₂O. RNA concentration and quality was determined using NanoDrop 2000 Spectrophotometer. RNA was stored at −80° C.

cDNA was synthesized using SuperScript™ IV VILO™ Master Mix with ezDNase™ Enzyme kit. For each reaction, 1500-1900 ng of RNA and were added. cDNA synthesis was performed according to protocol instructions supplied by kit provider. After synthesis cDNA was diluted in water to a final concentration of 20 ng/μland stored at −20° C.

### Quantitative RT-PCR

For each PCR reaction 10 μl Mastermix (2×), 2 μl TaqMan Gene expression assay and 20 ng of template cDNA were mixed in a total volume of 20 μl.

PCR was performed in a StepOnePLus™ Real-time PCR System instrument and analyzed by StepOne™Software v.2.3. The Comparative ΔΔCₜ method was used to quantify gene expression and expression of ribosomal protein L32 (RPL32) was used to normalize expression levels.

All reactions were performed in triplicates and non-template controls were included in each PCR run.

### Western Blot Analysis

Protein concentrations were determined using BCA protein assay kit. 10 or 20 μg of each protein lysate was boiled in 4× sample buffer with 2% β-mercaptoethanol and separated according to molecular weight on a 4-12% Bis-Tris Criterion XT Precast gel and electrophoretically transferred to a nitrocellulose membrane in a transfer buffer consisting of 25 mM Tris-HCl, 190 mM glycine, and 20% methanol. The membrane was blocked with 5% BSA in Tris-buffered saline with 0.1% Tween 20 (TBS/T) for 30 minutes at room temperature and then incubated over night at 4° C. with the primary antibodies diluted in blocking solution. Subsequently, the membranes were washed 4×5 minutes in TBS/T followed by incubation with horseradish peroxidase-conjugated secondary antibody diluted in 5% non-fat dried milk in TBS/T for one hour at room temperature. The membrane was washed 2×10 minutes in TBS/T before adding SuperSignal West Dura Extended duration substrate to visualize immunoreactive bands. The chemiluminescence signal was detected using a ChemiDoc imaging system.

### Results

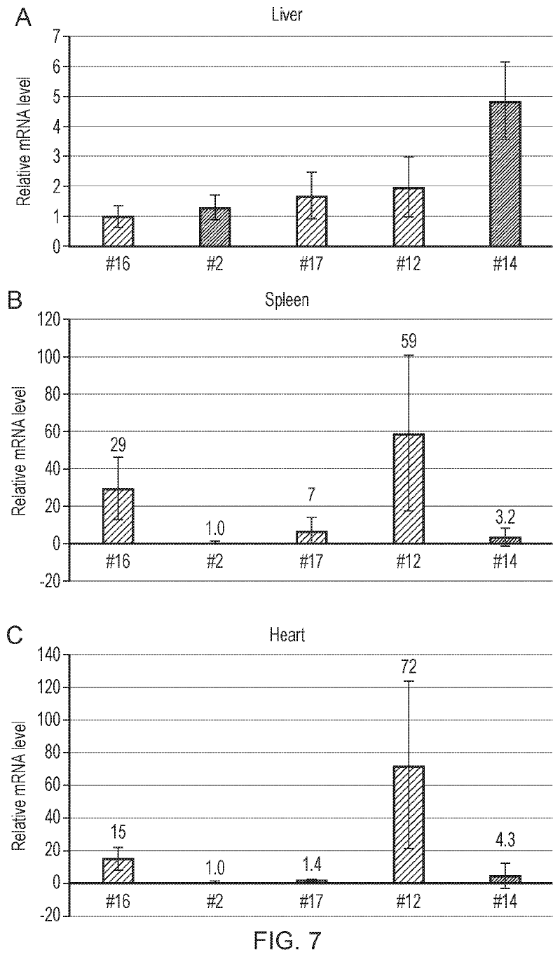

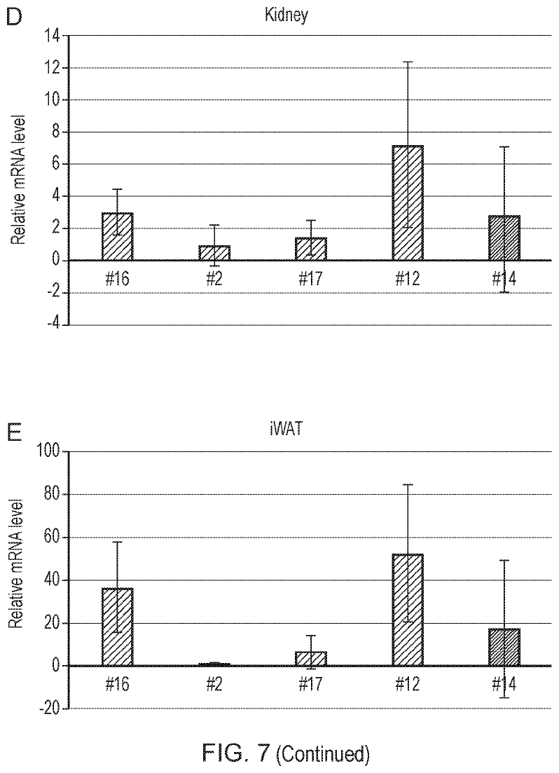

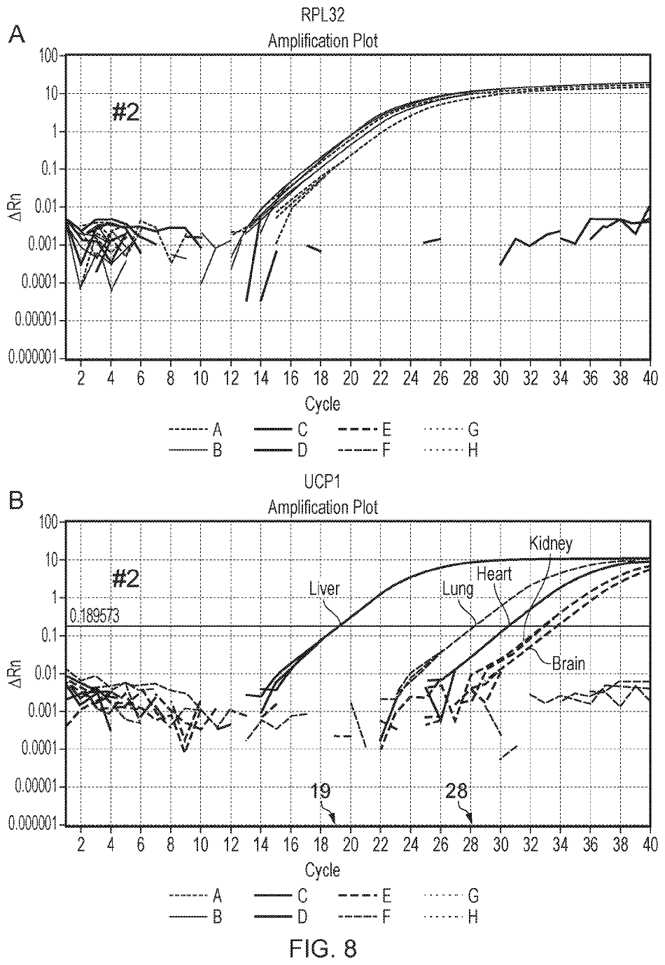

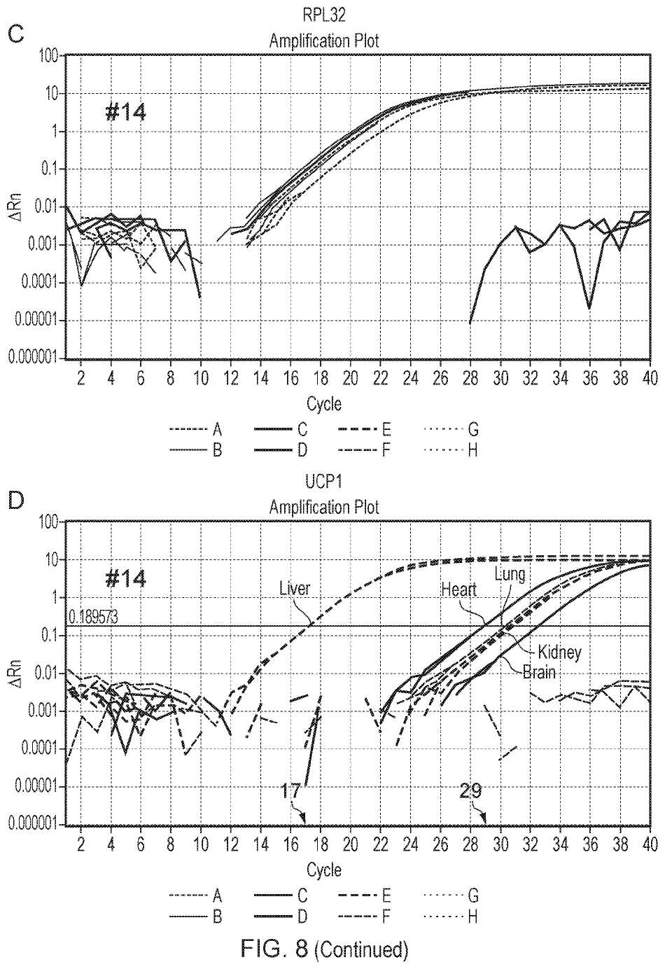

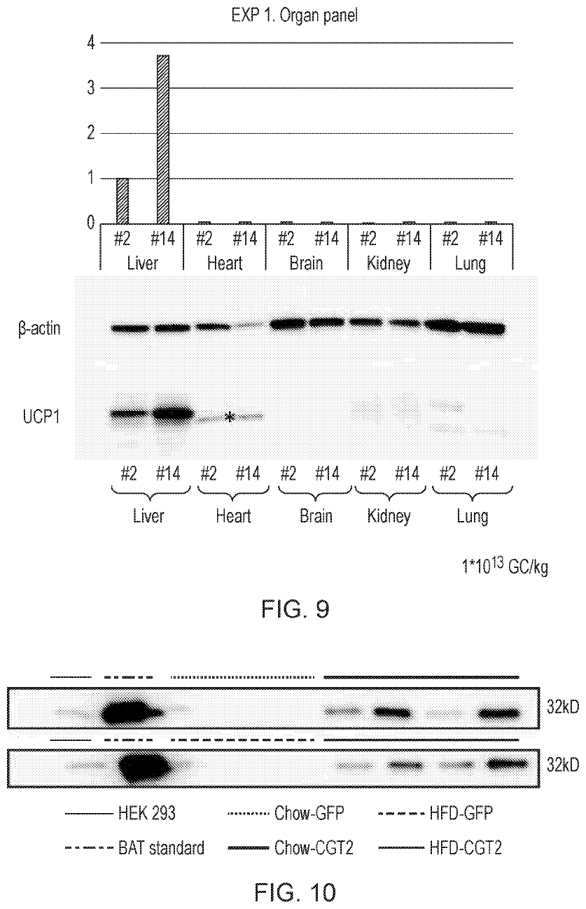

FIG. 7 shows liver specific expression from liver specific promoters/enhancers (i.e. vectors 2, 14 and 17). There is no/very low expression in other organs. Only vectors with CAG/Cbh promoters have expression in other organs (i.e. vectors 12 and 16). The highest expression in liver is from vector CGT2-14. FIGS. 8 and 9 also show the liver specific expression of vectors 2 and 14 with no/very low expression in other organs.

### Example 4

### Effect of Vectors on Body Fat

### Methods

Mice (C57BL/6J) were kept on chow or high fat diet (HFD) for 12 weeks and then were injected iv with vector CGT2-2 (dose 1×10¹³ vp/kg) or vector CGT2-14 (dose 1×10¹³) or with empty vector (control AAV-GFP dose 5×10¹² vp/kg) (n=4-7 each group). The mice were sacrificed after three weeks.

During these three weeks, the alive mice were monitored. In particular, their health status, activity, food intake, body weight, and body composition (fat and lean body mass with EchoMRI) were monitored. At the end of the exposure period, the organs (liver and adipose tissue depots) were collected, and wet weights were measured. Two small pieces of the median liver lobe were collected for triglyceride content analysis and UCP1 expression in whole tissue. The rest of the liver was taken for isolation of mitochondria. Most isolated fresh, intact liver mitochondria were functionally analyzed, while a small aliquot was saved in liquid nitrogen and used for Western blot.

### Results

UCP1 is targeted to mitochondria, and its protein level was identified in isolated liver mitochondria in all UCP1 injected animals, in contrast to control animals injected with empty vector (CFP). The high protein expression was evident for both vectors (CGT2-2 and CGT2-14) and on both diets (chow and high fat). Brown adipose tissue (BAT) mitochondria and HEK293 cells expressing native mouse and human UCP1 were used as standards for Western blot (see FIG. 10).

Functional analysis of liver mitochondria included parallel measurement of oxygen consumption and membrane potential. The oxygen consumption parameters were as follows: the basal oxygen consumption, the activity of the phosphorylation system, and the oxidative capacity. These parameters were analyzed on two types of substrates: Complex I (pyruvate 10 mM, glutamate 10 mM, malate 3 mM) and fatty acid-derived (Palmitoyl-CoA 25 μM, carnitine 10 mM and malate 3 mM).

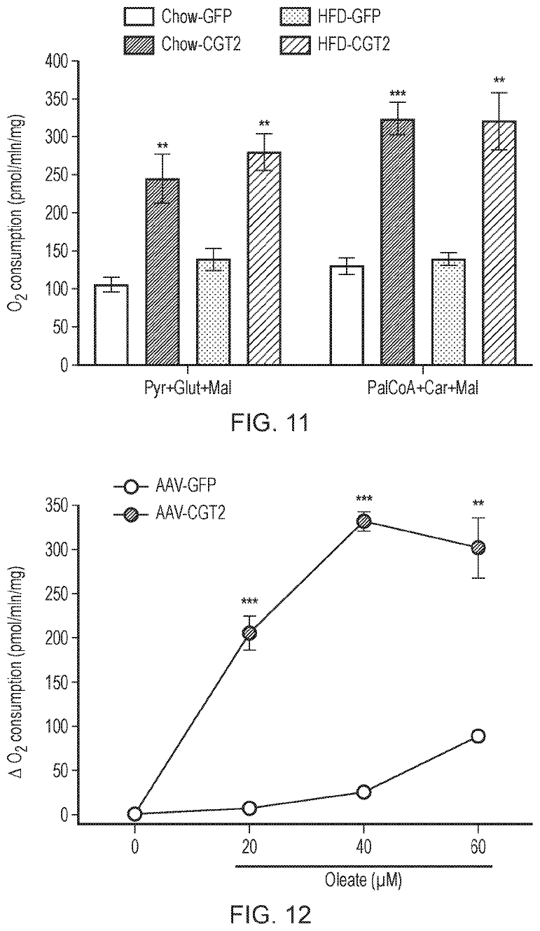

Based on the classical analysis of thermogenic brown adipose tissue mitochondria, functional UCP1 protein in mitochondria is expected to result in higher basal oxygen consumption. Indeed, markedly higher basal respiration was observed in liver mitochondria isolated from the UCP1-treated animals compared with control mitochondria (as expected). See FIG. 11. However, the rest of the oxygen consumption states were not significantly changed (also as expected).

Classically the unique feature of active thermogenic BAT mitochondria is the high sensitivity to natural uncoupling agents (fatty acids), and therefore such analysis has been performed to check the functionality of mitochondrially targeted UCP1. With functional protein presence in the mitochondria, UCP1 is expected to be more easily activated by fatty acids resulting in higher oxygen consumption. Indeed, significantly higher fatty acid sensitivity was shown here in mitochondria from mice treated with UCP1 compared with control GFP (See FIG. 12).

Parallel measurement of membrane potential under identical conditions revealed a significantly lower membrane potential in mitochondria isolated from mice treated with CGT2-2 and exposed to fatty acids (not shown), which corresponds to the oxygen consumption results (FIGS. 11 and 12). Such parallel changes in oxygen consumption and membrane potential (higher oxygen consumption vs. lower membrane potential in mitochondria) clearly reflect mitochondrial uncoupling due to functional UCP1.

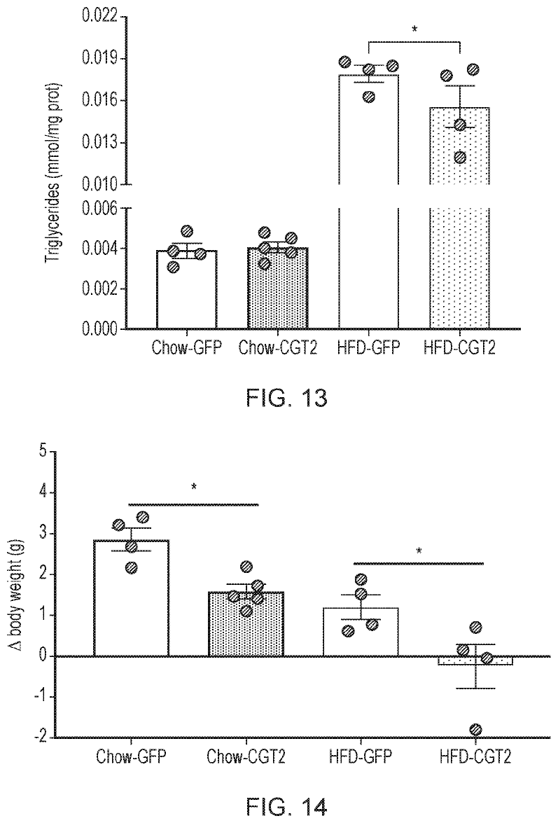

Thus, active UCP1 in mitochondria enhances the oxidation of substrates, including those derived from lipid sources. Such improved mitochondrial activity ultimately leads to reduced triglyceride levels in “fatty” liver (pathologically modified liver with significantly higher content of triglycerides from mice treated by high-fat diet) (FIG. 13).

Active UCP1 in the liver affected whole animal metabolism, leading to reduced delta body weight for UCP1 treated mice (FIG. 14). Notably, this positive effect on body weight is independent of food intake.

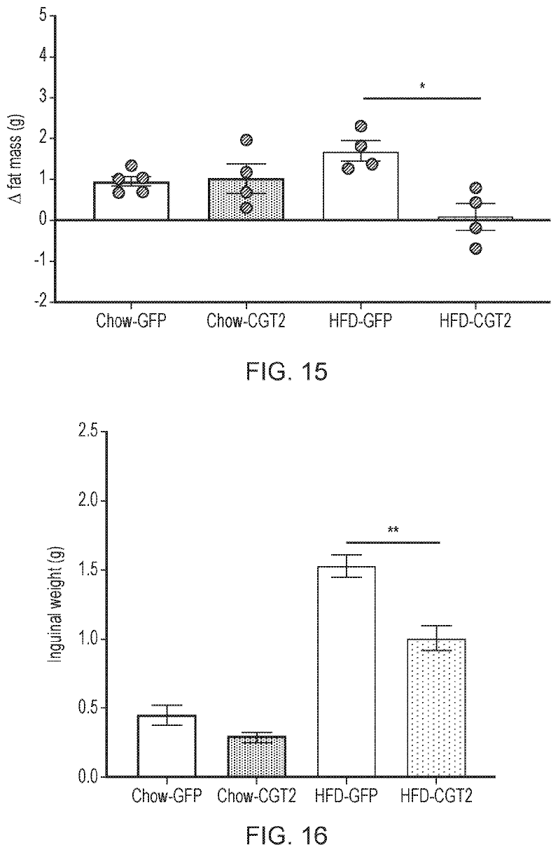

The reduction in delta body weight was mainly due to body fat mass (whereas lean body mass was not affected). Body fat reduction was evident both from measurements of body composition with magnetic resonance technique (EchoMRI), as well as wet tissue weights (FIG. 15 and FIG. 16).

### Example 5

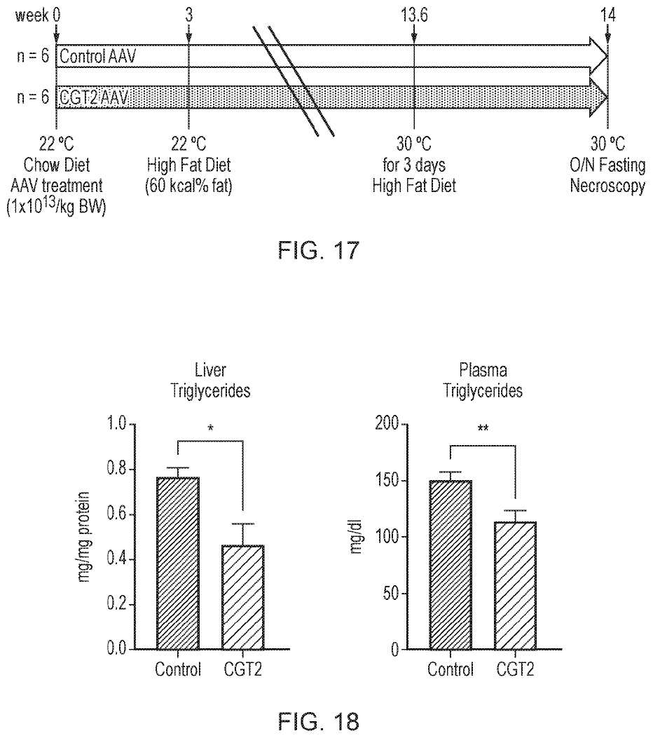

Female LDL receptor knockout mice (age 19-25 weeks) were treated with control or CGT2 AAV (6 per group). Starting three weeks after AAV treatment, the animals were kept on a high fat diet (60 cal % fat) for 11 weeks at 22° C. ambient temperature. Three days before organ harvest, the animals were kept at 30° C. ambient temperature. Before organ harvest the animals were fasted overnight. See FIG. 17. Blood was collected from the left ventricle of the heart into EDTA coated tubes. Tubes were placed on ice, centrifuged and plasma was isolated and assayed for triglycerides (TG) by using commercially available enzymatic kits from Roche Diagnostics (Mannheim, Germany). To determine hepatic TG concentration, 50 mg pieces of liver were homogenized by Tissue Lyzer (Qiagen) in a lysis buffer containing 2 mM CaCl₂, 80 mM NaCl, 1% TritonX-100, 50 mM Tris/HCl, pH 8.0. Triglycerides were determined using a standard enzyme-coupled colorimetric assay (Roche). Protein concentrations were measured by a Lowry method that was modified for lipid-containing samples by addition of 0.1% SDS. Results are presented in FIG. 18 as mg triglycerides/mg protein (liver triglycerides) and mg/dl (plasma triglycerides).

As shown in FIG. 18, the mice treated with UCP1 (CGT2) showed decreased liver triglycerides and decreased plasma triglycerides compared to the control. Student's t-test; *:p<0.05; **:p<0.01. Data is presented as means+/−SEM.

### Sequences

SEQ ID NO: 1—Human Uncoupling protein 1 (UCP1) nucleotide sequence (WT)
SEQ ID NO: 2—Human Uncoupling protein 1 (UCP1) protein sequence (WT)
SEQ ID NO: 3—CAG Promoter
SEQ ID NO: 4—CBh Promoter
SEQ ID NO: 5—TTR Promoter
SEQ ID NO: 6—hAAT Promoter
SEQ ID NO: 7—CRM8 enhancer
SEQ ID NO: 8—HCR enhancer
SEQ ID NO: 9—MVM intron
SEQ ID NO: 10—UCP1 intron 1
SEQ ID NO: 11—Woodchuck Hepatitis Virus post-transcriptional regulatory element
SEQ ID NO: 12—codon optimised human UCP1 gene
SEQ ID NO: 13—VECTOR 1A
SEQ ID NO: 14—VECTOR 1B
SEQ ID NO: 15—VECTOR 2
SEQ ID NO: 16—VECTOR 3
SEQ ID NO: 17—VECTOR 6
SEQ ID NO: 18—VECTOR 7
SEQ ID NO: 19—VECTOR 8
SEQ ID NO: 20—VECTOR 9
SEQ ID NO: 21—VECTOR 10
SEQ ID NO: 22—VECTOR 11
SEQ ID NO: 23—VECTOR 12
SEQ ID NO: 24—VECTOR 13
SEQ ID NO: 25—TBG Promoter

### REFERENCES

All patent and literature references cited in the present specification are hereby incorporated by reference in their entirety.
[1] Akinci B, et al., “Lipodystrophy Syndromes: Presentation and Treatment.,” [Online]. Available: [Updated 2018 Apr. 24]. In: Feingold K R, Anawalt B, Boyce A, et al., editors. Endotext [Internet]. South Dartmouth (MA): MDText.com, Inc.; 2000-. Available from: https://www.ncbi.nlm.nih.gov/books/NBK513130/.
[2] Lightbourne and Brown, “Genetics of Lipodystrophy,” Endocrinol Metab Clin North Am., vol. 2, no. 46, pp. 539-554, 2017.
[3] Bruder-Nascimento et al., “Recent advances in understanding lipodystrophy: a focus on lipodystrophy-associated cardiovascular disease and potential effects of leptin therapy on cardiovascular function,” F1000Research, vol. 8, no. 1756, 2019.
[4] Oral E A, et al, “Leptin-replacement therapy for lipodystrophy,” The New England Journal of Medicine., vol. 8, no. 346, p. 570-8.
[5] Busiello, R et al., “Mitochondrial uncoupling proteins and energy metabolism,” Front. Physiol, vol. 6, p. Article 36, 2015.
[6] Ishigaki et al., “Dissipating Excess Energy Stored in the Liver Is a Potential Treatment Strategy for Diabetes Associated with Obesity,” Diabetes, vol. 54, pp. 322-332, 2005.
[7] Chuah et al, “Liver-specific transcriptional modules identified by genome-wide in silico analysis enable efficient gene therapy in mice and non-human primates.,” Mol Ther., vol. 22, no. 9, pp. 1605-1613, 2014.
[8] Tews, D et al., “Elevated UCP1 levels are sufficient to improve glucose uptake in human white adipocytes”, Redox Biology 26 (2019) 101286.

1. A method for inhibiting a lipodystrophy in a subject, comprising:
administering a vector comprising a promoter operably linked to a nucleic acid encoding mitochondrial uncoupling protein 1 (UCP1) to a subject having lipodystrophy,
a. wherein the promoter is a liver specific promoter,
b. wherein the nucleic acid encoding UCP1 encodes a functional human UCP1 protein having the protein sequence of SEQ ID NO: 2, or a protein which has at least 90% sequence identity thereto, and
c. wherein an intron is included in the nucleic acid encoding UCP1 or before the UCP1 gene.

2-47. (canceled)

48. The method according to claim 1, wherein the vector is a lentivirus vector or an AAV vector.

49. The method according to claim 1, wherein the vector is selected from an AAV8 vector or an AAV3 vector.

50. The method according to claim 1, wherein the promoter is selected from the group consisting of a transthyretin (TTR) promoter, an alpha-1-antitrypsin promoter (hAAT) and a thyroxine binding globulin (TBG) promoter.

51. The method according to claim 1, wherein the vector further comprises an enhancer.

52. The method according to claim 51, wherein the enhancer is a CRM8 or an HCR enhancer.

53. The method according to claim 1, wherein the enhancer has the sequence of SEQ ID NO: 7 or SEQ ID NO: 8.

54. The method according to claim 1, wherein the nucleic acid encoding UCP1 comprises intron 1 from the UCP1 gene.

55. The method according to claim 1, wherein the vector is an AAV8 vector comprising a TBG or a TTR promoter operably linked to the nucleic acid encoding UCP1, wherein the vector further comprises a CRM8 enhancer, and wherein an intron is located between exons 1 and 2 of the nucleic acid encoding UCP1.

56. The method according to claim 1, wherein said vector is configured for gene therapy.

57. The method according to claim 1, wherein said lipodystrophy is a partial lipodystrophy.

58. The method according to claim 1, wherein said vector is incorporated into a pharmaceutical composition comprising one or more pharmaceutically acceptable excipients.

59. The method according to claim 1, wherein said vector is administered parenterally.

60. A host cell comprising:
a vector comprising a promoter operably linked to a nucleic acid encoding mitochondrial uncoupling protein 1 (UCP1), wherein the promoter is a liver specific promoter, wherein the nucleic acid encoding UCP1 encodes a functional human UCP1 protein having the protein sequence of SEQ ID NO: 2, or a protein that has at least 90% sequence identity thereto, and wherein an intron is included in the nucleic acid encoding UCP1 or before the UCP1 gene.

61. A nucleic acid comprising the nucleotide sequence set forth in SEQ ID NO: 12, or a nucleotide sequence with at least 75% identity thereto.
